(19) United States
(12) Patent Application Publication
(10) Pub. No.: US 2022/0175856 A1
(43) Pub. Date: Jun. 9, 2022

(54) UNRAVELING RECEPTOR-METABOLITE INTERACTIONS IN THE HUMAN MICROBIOME

(71) Applicant: The Rockefeller University, New York, NY (US)

(72) Inventor: Sean Brady, New York, NY (US)

(21) Appl. No.: 17/604,106

(22) PCT Filed: Apr. 20, 2020
(86) PCT No.: PCT/US20/28942

§ 371 Date: Oct. 15, 2021

Related U.S. Application Data
(60) Provisional application No. 62/836,309, filed on Apr. 19, 2019.

(51) Int. Cl. A61K 35/745; A61K 31/192; A61K 31/20; A61K 31/7088; A61K 35/747

(52) U.S. Cl. CPC A61K 35/745; A61K 31/192; A61K 35/747; A61K 31/7088; A61K 31/20

(57) ABSTRACT

The present invention relates to microorganisms and microbial metabolites derived therefrom that bind to a receptor and methods of use thereof

### STATEMENT REGARDING FEDERALLY SPONSORED RESEARCH OR DEVELOPMENT

This invention was made with government support under 5R01AT009562-02 awarded by the National Institutes of Health. The government has certain rights in the invention. sp

### CROSS-REFERENCE TO RELATED APPLICATIONS

This application claims priority under 35 U.S.C. §119(e) to U.S. Provisional Patent Application Ser. No. 62/836,309, filed Apr. 19, 2019, the disclosure of which is incorporated herein by reference in its entirety.

### BACKGROUND OF THE INVENTION

Human beings harbor dynamic and varied collections of bacteria that are thought to play important roles in health and disease. Despite evidence linking the human microbiome to health and disease, the mechanistic details of how the microbiota affects human physiology remain largely unknown (Cani PD, 2018, Gut 67:1716-1725). Much of the influence the microbiota has on its human host is likely encoded in the collection of small molecules it produces or modulates (Brown JM et al., 2017, J Biol Chem 292:8560-8568). Although global-omics approaches have uncovered correlations between aspects of the microbiota and biological outcomes, the mechanisms by which human associated bacteria influence host physiology remain poorly understood. Assigning function to these metabolites is critical to determining the molecular underpinnings of the host-microbe relationship and ultimately developing microbiota inspired therapies (Saha S et al., 2016, Drug Discov Today 21:692-698).

Furthermore, the majority of U.S. Food and Drug Administration (FDA)-approved drugs target just three receptor classes: GPCRs, ion channels, and nuclear hormone receptors (Santos R et al., 2017, Nat Rev Drug Discov 16:19-34). Many endogenous eukaryotic signaling metabolites target these same receptor families (Rosenbaum et al., 2009, Nature 459:356-363). Their dominant roles translating small molecules into biological responses suggested that these receptors will be important to the modulation of human physiology by metabolites encoded within the microbiota.

Thus, there is a need in the art for compositions and methods for identifying microbial metabolites that modulate GPCRs, and their host receptors. The present invention addresses this unmet need in the art.

### SUMMARY OF THE INVENTION

The present invention relates, in part, to a therapeutic composition comprising a microorganism and/or a microbial metabolite thereof. In various embodiments, the microorganism is Escherichia coli LF82, Enterococcus faecalis, Lactobacilhs plantarum, Faecalibacterium prauznitzii, Bifidobacterium longum, Bacteroides vulgatus, Ruminococcus gnavus, or any combination thereof.

In one embodiment, the microorganism binds to a receptor. In one embodiment, the microbial metabolite binds to a receptor. In various embodiments, the microorganism and/or the microbial metabolite thereof, binds to a receptor. In one embodiment, the receptor is at least one receptor listed in FIG. 2B and FIG. 15. In various embodiments, the receptor is a G-protein coupled receptor (GPCR), nuclear receptors (NR), or a combination thereof.

In various aspects of the invention, the microorganism and/or the microbial metabolite thereof modulates an activity level of a receptor by at least about 30%. In various embodiments, the microorganism and/or the microbial metabolite thereof increases the activity level of the receptor by at least about 30%. In various embodiments, the microorganism and/or the microbial metabolite thereof, inhibits the activity level of the receptor by at least about 30%.

In various embodiments, the microbial metabolite is a polypeptide, a carbohydrate, an oligosaccharide, a polysaccharide, a polynucleotide, a lipid, a phospholipid, a fatty acid, a steroid, a peptide, an amino acid, or any combination thereof. In various embodiments, the microbial metabolite is tyramine, tryptamine, cadaverine, 9,10-methylenehexadecanoic acid, 12-methyltetradecanoic acid (also referred to as 12-methylmyristic acid), 13-methyltetradecanoic acid (also referred to as 13-methylmyristic acid), 14-methylpalmitic acid, 4-hydroxycinnamic acid, anteiso-fatty acid, iso-fatty acid, or any combination thereof.

In one embodiment, tyramine binds to DRD1, DRD2L, DRD2S, DRD3, DRD4, DRD5, or any combination thereof. In one embodiment, tryptamine binds to HTR1A, HTR1B, HTR1F, HTR1E, HTR2A, HTR2C, HTR5A, or any combination thereof. In one embodiment, cadaverine binds to HRH1, HRH2, HRH3, HRH4, or any combination thereof. In one embodiment, 9,10-methylenehexadecanoic acid binds to BAIL CNR2, UTR2, or any combination thereof. In one embodiment, 12-methyltetradecanic acid binds to NMU1R, GPR151, UTR2, ADCYAP1R1, or any combination thereof. In one embodiment, 13-methyltetradecanic acid binds to GPR151. In one embodiment, 14-methylpalmitic acid binds to GPR151. In one embodiment, 4-hydroxycinnamic acid binds to GPR109B. In one embodiment, anteiso-fatty acids bind to NMU1R, UTR2, GPR120, or any combination thereof. In one embodiment, iso-fatty acid bind NMU1R, UTR2, GPR120, or any combination thereof.

In one aspect of the invention, the therapeutic composition is used to prevent or treat a disease or disorder in a subject in need thereof. In various embodiments, the disease or disorder is associated with dysfunction of at least one receptor listed in FIG. 2B and FIG. 15.

The present invention also relates, in part, to a method of treating a disease or disorder. In various embodiments, the methods of invention comprise administering any composition described herein to a subject in need thereof. In various embodiments, the disease or disorder is associated with dysfunction of at least one receptor listed in FIG. 2B and FIG. 15.

In various aspects of the invention, the methods of invention comprise administering a genetically engineered cell to a subject in need thereof. In one embodiment, the genetically engineered cell expresses a metabolite. In various embodiments, the metabolite binds to a receptor. In various embodiments, the receptor is at least one receptor listed in FIG. 2B and FIG. 15. In various embodiments, the microorganism is Escherichia coli LF82, Enterococcus faecalis, Lactobacilhs plantarum, Faecalibacterium prauznitzii, Bifidobacterium longum, Bacteroides vulgatus, Ruminococcus gnavus, or any combination thereof. In one embodiment, the microorganism or variants thereof is capable of producing a microbial metabolite. In various embodiment, the microbial metabolite is tyramine, tryptamine, cadaverine, 9,10-methylenehexadecanoic acid, 12-methyltetradecanoic acid (also referred to as 12-methylmyristic acid), 13-methyltetradecanoic acid (also referred to as 13-methylmyristic acid), 14-methylpalmitic acid, 4-hydroxycinnamic acid, anteiso-fatty acid, iso-fatty acid, or any combination thereof.

The present invention also relates, in part, to a method of treating a disease or disorder associated with at least one microorganism and/or a microbial metabolite. In various aspects of the invention, the methods of invention comprise modulating the level of the at least one microorganism and/or a microbial metabolite thereof in the subject.

In one embodiment, the method comprises reducing the level of the at least one microorganism and/or a microbial metabolite thereof in the subject. In one embodiment, the method comprises administering a therapeutically effective amount of an inhibitor of the at least one microorganism and/or a microbial metabolite thereof to the subject. In some embodiments, the inhibitor of the at least one microbial metabolite is an inhibitor of cadaverine, an inhibitor of cadaverine biosynthesis, or a combination thereof. In one embodiment, the disease or disorder associated with the at least one microorganism and/or a microbial metabolite thereof is a Crohn's disease.

In another embodiment, the method comprises increasing the level of the at least one microorganism and/or a microbial metabolite thereof in the subject.

The present invention relates, in part, to methods of screening and identifying a metabolite. In one embodiment, the metabolite is a microbial metabolite. In one embodiment, the microbial metabolite binds to a host receptor expressed by a host cell. In one embodiment, the method comprises measuring at least one activity of a host cell. In one embodiment, the method comprises contacting the host cell with at least one microbial metabolite test compound. In one embodiment, the method comprises measuring at least one activity of the host cell after it has been contacted with at least one microbial metabolite test compound. In one embodiment, the method comprises comparing the measurement of at least one activity of the host cell before it was contacted with at least one microbial metabolite test compound to the measurement of at least one activity of the host cell after it was contacted with at least one microbial metabolite test compound. In various embodiments, the method of identifying a microbial metabolite comprises measuring at least one activity of a host cell; contacting the host cell with at least one microbial metabolite test compound; measuring at least one activity of the host cell after it has been contacted with at least one microbial metabolite test compound; and comparing the measurement of at least one activity of the host cell before it was contacted with at least one microbial metabolite test compound to the measurement of at least one activity of the host cell after it was contacted with at least one microbial metabolite test compound.

In one embodiment, the measurement of at least one activity of the host cell before it was contacted with at least one microbial metabolite test compound is different than the measurement of at least one activity of the host cell after it was contacted with at least one microbial metabolite test compound. In one embodiment, the level of at least one activity of a host cell is elevated after the host cell is contacted with at least one microbial metabolite. In one embodiment, the level of at least one activity of a host cell is reduced after the host cell is contacted with at least one microbial metabolite. In various embodiments, the microbial metabolite test compound is identified as a microbial metabolite that binds to a host receptor.

In one embodiment, the host receptor is a G-protein coupled receptor (GPCR), nuclear receptors (NR), or a combination thereof. In one embodiment, the GPCR activity is reduced. In another embodiment, the GPCR activity is increased. In one embodiment, the GPCR is enriched in the gastrointestinal mucosa. In various embodiments, the GPCR is ADCYAP1R1, ADORA3, ADRA1B, ADRA2A, ADRA2B, ADRA2C, ADRBI, ADRB2, AGTRI, AGTRLI, AVPR1A, AVPR1B, AVPR2, BAIL BAI2, BAI3, BDKRB1, BDKRB2, BRS3, C3ARI, CSARI, C5L2, CALCR, CALCRL-RAMPI, CALCRL-RAMP2, CALCRL-RAMP3, CALCR-RAMP2, CALCR-RAMP3, CCKAR, CCKBR, CCR1, CCR10, CCR2, CCR3, CCR4, CCRS, CCR6, CCR7, CCR8, CCR9, CCRL2, CHRM1, CHRM2, CHRM3, CHRM4, CHRMS, CMKLRI, CNR1, CNR2, CRHR1, CRHR2, CRTH2, CX3CR1, CXCR1, CXCR2, CXCR3, CXCR4, CXCRS, CXCR6, CXCR7, DARC, DRD1, DRD2L, DRD2S, DRD3, DRD4, DRDS, EBI2, EDG1, EDG3, EDG4, EDGS, EDGE, EDG7, EDNRA, EDNRB, F2R, F2RL1, F2RL3, FFARI, FPRI, FPRLI, FSHR, G2A, GALR1, GALR2, GCGR, GHSR, GHSRIB, GIPR, GLP1R, GLP2R, GPR1, GPR101, GPR103, GPR107, GPR109A, GPR109B, GPR117, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37LI, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTRI, HCRTR2, HRH1, HRH2, HRH3, HRH4, HTR1A, HTR1B, HTR1E, HTR1F, HTR2A, HTR2C, HTRSA, KISSIR, LGR4, LGRS, LGR6, LHCGR, LTB4R, MC1R, MC3R, MC4R, MCSR, MCHR1, MCHR2, MLNR, MRGPRD, MRGPRE, MRGPRF, MRGPRX1, MRGPRX2, MRGPRX4, MTNRIA, NMBR, NMU1R, NPBWR1, NPBWR2, NPFFR1, NPSRIB, NPY1R, NPY2R, NTSR1, OPN5, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, VIPR2, or any combination thereof.

In one embodiment, the host cell is a human cell. In another embodiment, the host cell is an immune cell. In various embodiments, the host cell is an epithelial cell, a myeloid cell, a basophil, a neutrophil, an eosinophil, a monocyte, a macrophage, a natural killer (NK) cell, a macrophage, a dendritic cell (DC), a lymphocyte, an innate lymphoid cell (ILC), a B cell, a T cell, or any combination thereof.

In various embodiments, the activity is permeability, tight junction complex expression, mucus production, expression of an inflammatory cytokine, expression of an inflammatory chemokine, expression of an anti-inflammatory cytokines, expression of an anti-inflammatory chemokine, stimulation of a toll-like receptor (TLR), cell maturation, cell differentiation, cell activation, or any combination thereof.

In various embodiments, the microbial metabolite is a polypeptide, a carbohydrate, an oligosaccharide, a polysaccharide, a polynucleotide, a lipid, a phospholipid, a fatty acid, a steroid, a peptide, an amino acid, or any combination thereof. In various embodiments, the microbial metabolite is tyramine, tryptamine, cadaverine, 9,10-methylenehexadecanoic acid, 12-methyltetradecanoic acid (also referred to as 12-methylmyristic acid), 13-methyltetradecanoic acid (also referred to as 13-methylmyristic acid), 14-methylpalmitic acid, 4-hydroxycinnamic acid, anteiso-fatty acid, iso-fatty acid, or any combination thereof.

The present invention also relates, in part, to a composition comprising a microbial metabolite. In one aspect of the invention, the invention relates to a composition comprising a microbial metabolite variant. In another aspect of the invention, the invention relates to a composition comprising a microbial metabolite derivative. In various aspects of the invention, the invention relates to a composition comprising a microbial metabolite, microbial metabolite variant, microbial metabolite derivative, or any combination thereof. In various embodiments, the microbial metabolite is identified using any of the methods described herein. In various embodiments, the microbial metabolite variant is identified using any of the methods described herein. In various embodiments, the microbial metabolite derivative is identified using any of the methods described herein.

The present invention also discloses methods of identifying a host cell receptor. In one embodiment, the host cell receptor binds to a microbial metabolite. In one embodiment, the method comprises identifying a candidate host cell receptor. In one embodiment, the method comprises obtaining a genetically modified cell. In one embodiment, the method comprises contacting the genetically modified cell that expresses the candidate host cell receptor with at least one microbial metabolite test compound. In one embodiment, the genetically modified cells is genetically modified to express the candidate host cell receptor. In one embodiment, the candidate host cell receptor is tagged with a detectable marker. In one embodiment, the detectable marker is detected. In one embodiment, the method comprises detecting the presence of the detectable marker. In various embodiments, the method comprises identifying a candidate host cell receptor; obtaining a genetically modified cell; contacting the genetically modified cell that expresses the candidate host cell receptor with at least one microbial metabolite test compound; detecting the presence of the detectable marker, wherein when the detectable marker is detected, the candidate host receptor is identified as a host cell receptor that binds to a microbial metabolite. In one embodiment, the candidate host receptor is identified as a host cell receptor that binds to a microbial metabolite.

In one embodiment, at least one microbial metabolite is a known microbial metabolite. In one embodiment, at least one microbial metabolite is an unknown microbial metabolite. In one embodiment, at least one microbial metabolite is mixture of unknown microbial metabolites. In various embodiments, the microbial metabolite is a polypeptide, a carbohydrate, an oligosaccharide, a polysaccharide, a polynucleotide, a lipid, a phospholipid, a fatty acid, a steroid, a peptide, an amino acid, or any combination thereof. In various embodiments, the microbial metabolite is tyramine, tryptamine, cadaverine, 9,10-methylenehexadecanoic acid, 12-methyltetradecanoic acid (also referred to as 12-methylmyristic acid), 13-methyltetradecanoic acid (also referred to as 13-methylmyristic acid), 14-methylpalmitic acid, 4-hydroxycinnamic acid, anteiso-fatty acid, iso-fatty acid, or any combination thereof.

In various embodiments, the detectable marker is an antibiotic resistance gene, a gene encoding a fluorescent protein, or a combination thereof. In various embodiments, the candidate host receptor is a GPCR, NR, or a combination thereof. In one embodiment, the GPCR activity is reduced. In another embodiment, the GPCR activity is increased. In various embodiments, the GPCR is ADCYAP1R1, ADORA3, ADRA1B, ADRA2A, ADRA2B, ADRA2C, ADRBI, ADRB2, AGTRI, AGTRLI, AVPR1A, AVPR1B, AVPR2, BAI1, BAI2, BAI3, BDKRB1, BDKRB2, BRS3, C3ARI, C5ARI, C5L2, CALCR, CALCRL-RAMPI, CALCRL-RAMP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLRI, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRDS, EBI2, EDG1, EDG3, EDG4, EDGS, EDGE, EDG7, EDNRA, EDNRB, F2R, F2RL1, F2RL3, FFARI, FPRI, FPRLI, FSHR, G2A, GALR1, GALR2, GCGR, GHSR, GHSRIB, GIPR, GLP1R, GLP2R, GPR1, GPR101, GPR103, GPR107, GPR109A, GPR109B, GPR117, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37LI, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTRI, HCRTR2, HRH1, HRH2, HRH3, HRH4, HTR1A, HTR1B, HTR1E, HTR1F, HTR2A, HTR2C, HTRSA, KISSIR, LGR4, LGRS, LGR6, LHCGR, LTB4R, MC1R, MC3R, MC4R, MCSR, MCHR1, MCHR2, MLNR, MRGPRD, MRGPRE, MRGPRF, MRGPRX1, MRGPRX2, MRGPRX4, MTNRIA, NMBR, NMU1R, NPBWR1, NPBWR2, NPFFR1, NPSRIB, NPY1R, NPY2R, NTSR1, OPNS, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTRS, TAARS, TACR1, TACR2, TACR3, TBXA2R, TRHR, TSHR(L), UTR2, VIPR1, VIPR2, or any combination thereof.

In one embodiment, the genetically modified cell is a human cell. In one embodiment, the genetically modified cell is CHO, BHK, HEK293, VERO, HeLa, COS, MDCK, NSO, W138, or any combination thereof.

The present invention also relates, in part, to a composition comprising a host receptor. In another aspect of the invention, the invention relates to a composition comprising a host receptor variant. In another aspect of the invention, the invention relates to a host receptor derivative. In various embodiments, the host receptor is identified using any of the methods described herein. In various embodiments, the host receptor variant is identified using any of the methods described herein. In various embodiments, the host receptor derivative is identified using any of the methods described herein.

The present invention further discloses methods of screening a drug that binds to a host receptor expressed by a host cell. In one embodiment, the method comprises identifying a microbial metabolite that binds to a host receptor expressed by a host cell. In one embodiment, the method comprises measuring at least one activity of a host cell. In one embodiment, the method comprises contacting the host cell with at least one drug test compound. In one embodiment, the method comprises contacting the host cell with at least one microbial metabolite. In one embodiment, the method comprises measuring at least one activity of the host cell after it has been contacted with at least one drug test compound. In one embodiment, the method comprises measuring at least one activity of the host cell after it has been contacted with at least one microbial metabolite. In one embodiment, the method comprises comparing the measurement of at least one activity of the host cell before it was contacted with at least one drug test compound to the measurement of at least one activity of the host cell after it was contacted with at least one drug test compound and/or at least one microbial metabolite. In one embodiment, the measurement of at least one activity of the host cell before it was contacted with at least one drug test compound is different than the measurement of at least one activity of the host cell after it was contacted with at least one drug test compound and/or at least one microbial metabolite. In one embodiment, the drug test compound is identified as a drug that binds to a host receptor. In various embodiments, the method comprises identifying a microbial metabolite that binds to a host receptor expressed by a host cell; measuring at least one activity of a host cell; contacting the host cell with at least one drug test compound; contacting the host cell with at least one microbial metabolite; measuring at least one activity of the host cell after it has been contacted with at least one drug test compound; measuring at least one activity of the host cell after it has been contacted with at least one microbial metabolite; and comparing the measurement of at least one activity of the host cell before it was contacted with at least one drug test compound to the measurement of at least one activity of the host cell after it was contacted with at least one drug test compound and/or at least one microbial metabolite.

The present invention relates, in part, to methods of screening a drug that binds to a microbial metabolite. In one embodiment, the method comprises identifying a microbial metabolite that binds to a host receptor expressed by a host cell. In one embodiment, the method comprises measuring at least one activity of a host cell. In one embodiment, the method comprises contacting at least one microbial metabolite with at least one drug test compound. In one embodiment, the method comprises contacting the host cell with at least one drug test compound and at least one microbial metabolite. In one embodiment, the method comprises measuring at least one activity of the host cell after it has been contacted with at least one drug test compound and at least one microbial metabolite. In one embodiment, the method comprises comparing the measurement of at least one activity of the host cell after it was contacted with at least one microbial metabolite and before it was contacted with at least one drug test compound to the measurement of at least one activity of the host cell after it was contacted with at least one drug test compound and at least one microbial metabolite. In one embodiment, the measurement of at least one activity of the host cell after it was contacted with at least one microbial metabolite and before it was contacted with at least one drug test compound is different than the measurement of at least one activity of the host cell after it was contacted with at least one drug test compound and at least one microbial metabolite. In one embodiment, the drug test compound is identified as a drug that binds to a microbial metabolite. In various embodiments, the method comprises identifying a microbial metabolite that binds to a host receptor expressed by a host cell; measuring at least one activity of a host cell; contacting at least one microbial metabolite with at least one drug test compound; contacting the host cell with at least one drug test compound and at least one microbial metabolite; measuring at least one activity of the host cell after it has been contacted with at least one drug test compound and at least one microbial metabolite; and comparing the measurement of at least one activity of the host cell after it was contacted with at least one microbial metabolite and before it was contacted with at least one drug test compound to the measurement of at least one activity of the host cell after it was contacted with at least one drug test compound and at least one microbial metabolite.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts experimental procedure for generating library of secreted bacterial metabolites from large-scale monocultures of bacteria.

FIG. 2, comprising FIG. 2A and FIG. 2B, depicts heat map of individual assays for each GPCR tested and the subset of GPCRs. FIG. 2A depicts heat map of individual assays for each GPCR tested indicating β-arrestin recruitment response normalized to endogenous or synthetic control compound (100%). FIG. 2B depicts subset of GPCRs demonstrating <30% response to media control, while having >30% response to bacterial fractions. GPCRs in green are designated with orphan status. Expression of receptors in tissues commonly exposed to human, as reported in the Human Protein Atlas (Uhlén M et al., 2015, Science 347:6220). Receptors targeted by approved FDA drugs indicated below (Sriram K et al., 2018, Insel).

FIG. 3 depicts heat map depicting agonism of select neurotransmitter receptor families, the serotonin receptors (HTRs), dopamine receptors (DRDs), and histamine receptors (HRHs).

FIG. 4 depicts structures of active compounds alongside dose response curves of synthetic compounds for respective GPCRs.

FIG. 5 depicts heat map of GPCRs demonstrating general (top) or specific (bottom) responses to lipid-rich fractions of bacterial extracts. Red indicates bacterial species in which specific lipid responses were present. Raw HPLC-charged aerosol detector (CAD) chromatograms for each lipid fraction.

FIG. 6 depicts overlaid CAD chromatograms with common lipids and unique lipids (red asterisk) marked.

FIG. 7 depicts structure of BAIl-active lipid 9,10-methylenehexadecanoic acid isolated from E. coli LF82 and response of BAI1 to various fatty acids.

FIG. 8 depicts structure of NMU1R-active lipid, 12-methylmyristic acid, isolated from B. vulgatus and response of NMU1R GPCR to various fatty acids.

FIG. 9 depicts panel of branched chain fatty acids tested for GPCR fidelity.

FIG. 10 depicts response of GPR120 (general), NMU1R, and UTR2 (specific) to branched chain fatty acid panel.

FIG. 11 depicts schematic of murine experiments to test the production of bacterial metabolites in vivo.

FIG. 12 depicts relative quantitative analysis of metabolites using high-resolution mass spectrometry, n=6, error bars are SD.

FIG. 13, comprising FIG. 13A through FIG. 13C, depicts representative results demonstrating that cadaverine is an HRH4 agonist, is secreted by pathogenic bacteria, and influences gut inflammation. FIG. 13A depicts representative results demonstrating that cadaverine agonizes inflammatory receptor and is increased in Crohn's disease (CD) patients. FIG. 13B depicts representative results demonstrating that cadaverine production is regulated by acid response and can be genetically decreased. FIG. 13C depicts representative results demonstrating that CadA influence colitis phenotypes in vivo. FIG. 13C depicts representative results demonstrating the pathohistology of cecum (1), colon length after dextran sulfate sodium (DSS) tx (2), weight loss during DSS tx (3), and fecal inflammation marker (lcn-2) of IL10^{-/-} mice (4).

FIG. 14 depicts a schematic representation demonstrating that cadaverine production is dictated by taxa specific cadA gene, which is prevalent in pathogenic bacteria.

FIG. 15, comprising FIG. 15A through FIG. 15C, depicts representative results demonstrating that fatty acids are oft-understudied but important bacterial bioactives. FIG. 15A depicts representative results demonstrating that certain GPCRs were highly responsive to fatty acid-rich fractions of bacteria. FIG. 15B depicts representative results demonstrating that specific lipids activated specific GPCRs. FIG. 15C depicts representative results demonstrating that bacterial specific lipids help explain health discrepancies in human microbiome.

FIG. 16, comprising FIG. 16A through FIG. 16C, depicts representative results demonstrating murine models to study effects of taxa-specific fatty acids. FIG. 16A depicts schematic representation demonstrating pipeline for analyzing bacterial fatty acids in vivo. FIG. 16B depicts representative results demonstrating that cyclopropyl lipids agonized orphan and established immune GPCRs. FIG. 16C representative results demonstrating the immune responses seen in mice tissue in response to cyclopropyl lipid production.

### DETAILED DESCRIPTION

The present invention relates to assays and methods for screening microbial metabolite test compounds to identify microbial metabolite compounds that bind to a host receptor. The present invention also relates to assays and methods for screening and identifying host receptors by their ability to bind to a microbial metabolite. In some embodiments of the assays and methods of the invention, the microbial metabolite test compound is a known microbial metabolite. In other embodiments of the assays and methods of the invention, the microbial metabolite test compound is an unknown microbial metabolite. In some embodiments of the assays and methods of the invention, the host receptor is a receptor that is known to bind to a microbial metabolite. In other embodiments of the assays and methods of the invention, the host receptor is a receptor that is not known to bind to a microbial metabolite. In some embodiments, the microbial metabolite is an agonist of the host receptor. In other embodiments, the microbial metabolite is an antagonist of the host receptor. The present invention also relates, in part, to methods of treating or preventing disease or disorder associated with at least one microorganism and/or a microbial metabolite of the present invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “agent” includes any substance, metabolite, molecule, element, compound, entity, or a combination thereof. It includes, but is not limited to, e.g., protein, oligopeptide, small organic molecule, polysaccharide, polynucleotide, and the like. It can be a natural product, a synthetic compound, a chemical compound, or a combination of two or more substances. Unless otherwise specified, the terms “agent,” “substance,” and “compound” can be used interchangeably. Further, a “test agent” or “candidate agent” is generally a subject agent for use in an assay of the invention.

The term “assessing” includes any form of measurement, and includes determining if an element is present or not. The terms “determining,” “measuring,” “evaluating,” “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent). “Assessing activity” includes determining the amount of activity, and/or determining whether an activity has occurred (i.e., whether an activity is present or absent).

The term “binding” refers to a direct association between at least two molecules, due to, for example, covalent, electrostatic, hydrophobic, ionic and/or hydrogen-bond interactions.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components. Alternatively, molecules or components thereof may be contacted combining a fluid component with molecules immobilized on or in a cell or on or in a substrate, such as a polymer bead, a membrane, a polymeric glass substrate or substrate surface derivatized to provide immobilization of target molecules, candidate molecules, competitive binding reference molecules or any combination of these. Molecules or components thereof may be contacted by selectively adjusting solution conditions such as, the composition of the solution, ion strength, pH or temperature. Molecules or components thereof may be contacted in a static vessel, such as a microwell of a microarray system, or a flow-through system, such as a microfluidic or nanofluidic system. Molecules or components thereof may be contacted in or on a variety of cells, media, liquids, solutions, colloids, suspensions, emulsions, gels, solids, membrane surfaces, glass surfaces, polymer surfaces, vesicle samples, bilayer samples, micelle samples and other types of cellular models or any combination of these.

“Non-pathogenic bacteria” refer to bacteria that are not capable of causing disease or harmful responses in a host. In some embodiments, non-pathogenic bacteria are commensal bacteria. Examples of non-pathogenic bacteria include, but are not limited to Escherichia coli LF82, Enterococcus faecalis, Lactobacillis plantarum, Faecalibacterium prauznitzii, Bifidobacterium longum, Bacteroides vulgatus, Ruminococcus gnavus, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium in/antis, Bifidobacterium lactis, Bifidobacterium longum, Clostridium butyricum, Enterococcus /aecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, a “prebiotic” is a selectively fermented ingredient that allows specific changes, both in the composition and/or activity in the gastrointestinal microflora, which confers benefits upon host well-being and health.

As used herein, the term “diagnosis” refers to the determination of the presence of a disease or disorder. In some embodiments of the present invention, methods for making a diagnosis are provided which permit determination of the presence of a particular disease or disorder.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A disease or disorder is “alleviated” if the severity of a symptom of the disease or disorder, the frequency with which such a symptom is experienced by a patient, or both, is reduced.

As used herein, “treating a disease or disorder” means reducing the frequency with which a sign or symptom of the disease or disorder is experienced by a patient.

A “therapeutic” treatment is a treatment administered to a subject who exhibits signs or symptoms of pathology, for the purpose of diminishing or eliminating those signs or symptoms.

The phrase “effective amount” or “therapeutically effective amount,” as used herein, refers to an amount that is sufficient or effective to prevent or treat (delay or prevent the onset of, prevent the progression of, inhibit, decrease or reverse) a disease or disorder. An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

As used herein, the terms “downstream” or “upstream” with respect to a signaling pathway is based on epistatic relationships in a linear signaling cascade: if “A” activates “B” and “B” activates “C”, the “A” is upstream of “B” and “B” is upstream of “C”. Similarly, “B” is downstream of “A” and “C” is downstream of “B”.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (e.g., horseradish peroxidase, beta-galactosidase, luciferase, alkaline phosphatase), biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

“G protein-coupled receptors” (GPCRs) are also known as seven transmembrane receptors, 7TM receptors, heptahelical receptors, and G protein linked receptors (GPLR). GPCRs are a large protein family of transmembrane receptors that sense molecules outside the cell and activate inside signal transduction pathways and, ultimately, cellular responses. The ligands that bind and activate these receptors include light-sensitive compounds, odors, pheromones, hormones, and neurotransmitters, and vary in size from small molecules to peptides to large proteins. GPCRs are involved in many diseases, but are also the target of around half of all modern medicinal drugs. GPCRs can be grouped into 4 classes based on sequence homology and functional similarity: Class A rhodopsin-like, Class B secretin-like, Class C metabotropic/pheromone, and Class D fungal pheromone. GPCRs are involved in a wide variety of physiological processes, including the visual sense, the sense of smell, behavioral and mood regulation, regulation of immune system activity and inflammation, autonomic nervous system transmission, cell density sensing, and many others. GPCRs include receptors for sensory signal mediators (e.g., light and olfactory stimulatory molecules); adenosine, bombesin, bradykinin, endothelin, y-aminobutyric acid (GABA), hepatocyte growth factor, melanocortins, neuropeptide Y, opioid peptides, opsins, somatostatin, tachykinins, vasoactive intestinal polypeptide family, and vasopressin; biogenic amines (e.g., dopamine, epinephrine and norepinephrine, histamine, glutamate (metabotropic effect), acetylcholine (muscarinic effect), and serotonin); chemokines; lipid mediators of inflammation (e.g., prostaglandins and prostanoids, platelet activating factor, and leukotrienes); and peptide hormones (e.g., calcitonin, C5a anaphylatoxin, follicle stimulating hormone (FSH), gonadotropic-releasing hormone (GnRH), neurokinin, and thyrotropin releasing hormone (TRH), and oxytocin). GPCRs which act as receptors for stimuli that have yet to be identified are known as orphan receptors. It is known that the inactive G protein is bound to the receptor in its inactive state. Once the ligand is recognized, the receptor shifts conformation and thus mechanically activates the G protein, which detaches from the receptor. The receptor can now either activate another G protein, or switch back to its inactive state. This is an overly simplistic explanation, but suffices to convey the overall set of events. It is believed that a receptor molecule exists in a conformational equilibrium between active and inactive biophysical states. The binding of ligands to the receptor may shift the equilibrium toward the active, or the inactive, receptor states. A GPCR can mediate both G protein-dependent and independent signaling, often in a ligand-dependent manner.

As used herein, an “inhibitory-effective amount” is an amount that results in a detectable (e.g., measurable) amount of inhibition of an activity. In some instance, the activity is its ability to bind with another component.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

In the context of the present disclosure, a “modulator” is defined as a compound that is an agonist, a partial agonist, an inverse agonist or an antagonist of a GPCR, microorganism, microbial metabolite thereof, or any combination thereof. A modulator may increase the activity of the GPCR, microorganism, microbial metabolite thereof, or any combination thereof, or may decrease the activity of the GPCR, microorganism, microbial metabolite thereof, or any combination thereof.

In the context of the present disclosure, an “agonist” is defined as a compound that increases the basal activity of a receptor (i.e. signal transduction mediated by the receptor). The term “agonist” as used herein refers to a drug which binds to a receptor and activates it, producing a pharmacological response (contraction, relaxation, secretion, enzyme activation, etc.).

An “antagonist” is defined as a compound, which blocks the action of an agonist on a receptor. A “partial agonist” is defined as an agonist that displays limited, or less than complete, activity such that it fails to activate a receptor in vitro, functioning as an antagonist in vivo. The term “antagonist” as used herein refers to a drug which attenuates the effect of an agonist. It may be competitive (or surmountable), i.e. it binds reversibly to a region of the receptor in common with an agonist, but occupies the site without activating the effector mechanism. The effects of a competitive antagonist may be overcome by increasing the concentration of agonist, thereby shifting the equilibrium and increasing the proportion of receptors which the agonist occupies. However, it is now known that certain antagonists can affect receptor trafficking and therefore improve agonist actions indirectly.

An “inverse agonist” is defined as a compound that decreases the basal activity of a receptor.

The term “interact” or “interaction” refers to a measurable chemical or physical interaction between two components, such as a target molecule and a candidate molecule, that is capable of affecting the structure and/or composition of at least one of the components, such as a target molecule, a candidate molecule or both such that the biological activity of at least one of the components, such as the target molecule, the candidate molecule or both, is affected. Interactions capable of affecting the structure and/or composition of a component include, but are not limited to, reactions resulting in the formation of one or more covalent bonds, resulting in the breaking of one or more covalent bonds, electrostatic associations and repulsions, formation and/or disruption of hydrogen bonds, formation and/or disruption of electrostatic forces such as dipole-dipole interactions, formation and/or disruption of van der Waals interactions or processes comprising combinations of these.

“Microbial metabolite” refers to any compound produced by or derived from a microbe. Non-limiting examples include proteins, peptides, lipids, DNA molecules, RNA molecules, oligonucleotides, carbohydrates, polysaccharides, glycoproteins, lipoproteins, sugars, amino acids, and carboxylic acids, as well as derivatives, variants and complexes of these.

“Molecule” refers to a collection of chemically bound atoms with a characteristic composition. As used herein, a molecule can be neutral or can be electrically charged. The term molecule includes biomolecules, which are molecules that are produced by an organism or are important to a living organism, including, but not limited to, proteins, peptides, lipids, DNA molecules, RNA molecules, oligonucleotides, carbohydrates, polysaccharides, glycoproteins, lipoproteins, sugars and derivatives, variants and complexes of these, including labeled analogs of these having one or more vibrational tag. The term molecule also includes candidate molecules, which comprise any molecule that it is useful, beneficial or desirable to probe its capable to interact with a molecule such as a target molecule. Candidate molecules include therapeutic candidate molecules which are molecules that may have some effect on a biological process or series of biological processes when administered. Therapeutic candidate molecules include, but are not limited to, drugs, pharmaceuticals, metabolites, potential drug candidates and metabolites of drugs, biological therapeutics, potential biological therapeutic candidates and metabolites of biological therapeutics, organic, inorganic and/or hybrid organic-inorganic molecules that interact with one or more biomolecules, molecules that inhibit, decrease or increase the bioactivity of a biomolecule, inhibitors, ligands and derivatives, variants and complexes of these. The term molecule also includes target molecules, which comprise any molecule that it is useful, beneficial or desirable to probe its capable to interact with a molecule such as a candidate molecule. Target molecules useful for identifying, characterizing and/or optimizing therapeutics and therapeutic candidates comprise biomolecules, and derivatives, variants and complexes of biomolecules. The term molecule also includes competitive binding reference molecules. Competitive binding reference molecules useful in the present invention are molecules that are known to bind, at least to some extent, to a target molecule, and in some embodiments comprise a known drug, biological therapeutic, biomolecule, lead compound in a drug discovery program, and derivatives, variants, metabolites and complexes of these.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein “polymorph” refers to crystalline forms having the same chemical composition but different spatial arrangements of the molecules, atoms, and/or ions forming the crystal.

The term “pharmaceutically acceptable salt” refers to any pharmaceutically acceptable salt, which upon administration to the patient is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

“Screening” referred to in the present invention includes not only so-called first screening for identifying a compound of the present invention among a plurality of candidate compounds, but also a counter screen for identifying a compound of the present invention among a plurality of candidate compounds.

As used herein, “substantially pure” means an object species is the predominant species present (i.e., it is the most abundant of any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

“Test agents” or otherwise “test compounds” as used herein refers to an agent or compound that is to be screened in one or more of the assays described herein. Test agents include compounds of a variety of general types including, but not limited to, metabolites, small organic molecules, known pharmaceuticals, polypeptides, carbohydrates (such as oligosaccharides and polysaccharides), polynucleotides, lipids, phospholipids, fatty acids, steroids, peptides, amino acids, or amino acid analogs. Test agents can be obtained from microbial culture supernatants or microbial culture lysates. Test agents can also be obtained from libraries, such as natural product libraries and combinatorial libraries. In addition, methods of automating assays are known that permit screening of several thousands of compounds in a short period.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“Variant” as the term is used herein, is a molecule that differs in structure from a reference molecule, but retains essential physical or biological properties of the reference molecule. “Variant” as the term is used herein, is also a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

A “fragment” of a nucleic acid sequence that encodes an antigen may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antigen and additionally optionally comprise sequence encoding an N terminal methionine or heterologous signal peptide which is not included when calculating percent identity.

As used herein, an “instructional material” includes a publication, a recording, a diagram, or any other medium of expression which can be used to communicate the usefulness of a compound, composition, method or assay of the invention in the kit. The instructional material of the kit of the invention can, for example, be affixed to a container which contains the identified compound, composition, method or assay of the invention or be shipped together with a container which contains the identified compound, composition, method or assay of the invention. Alternatively, the instructional material can be shipped separately from the container with the intention that the instructional material and the compound be used cooperatively by the recipient.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to assays and methods for screening microbial metabolite test compounds or test drug compounds to identify microbial metabolite compounds or drug compounds that bind to a host receptor. The present invention also relates to assays and methods for screening and identifying host receptors or drug compounds by their ability to bind to microbial metabolites. In some embodiments, the host is a mammal. In preferred embodiments, the host is a human. The present invention also relates, in part, to methods of treating or preventing disease or disorder associated with at least one microorganism and/or a microbial metabolite of the present invention.

### Composition

In one embodiment, the invention provides a therapeutic composition comprising a microorganism and/or a microbial metabolite derived thereof In various embodiments, the microorganism is Escherichia coli LF82, Enterococcus faecalis, Lactobacilhs plantarum, Faecalibacterium prauznitzii, Bifidobacterium longum, Bacteroides vulgatus, Ruminococcus gnavus, or any combination thereof. However, the invention should not be limited to these microorganisms. Rather, the invention includes any microorganism that secretes or otherwise produces the desired metabolite discussed elsewhere herein.

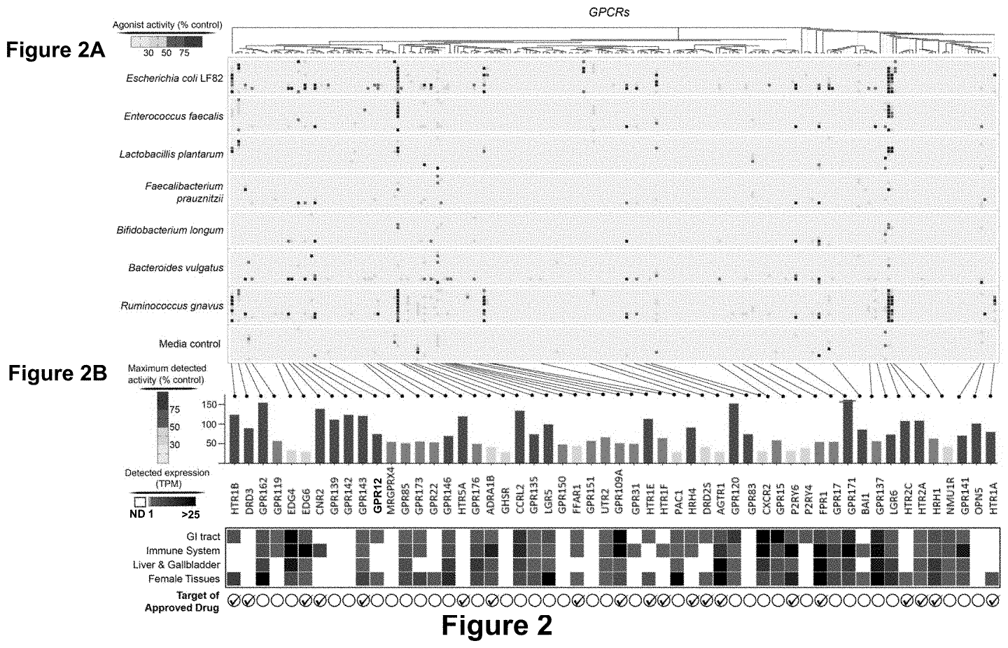

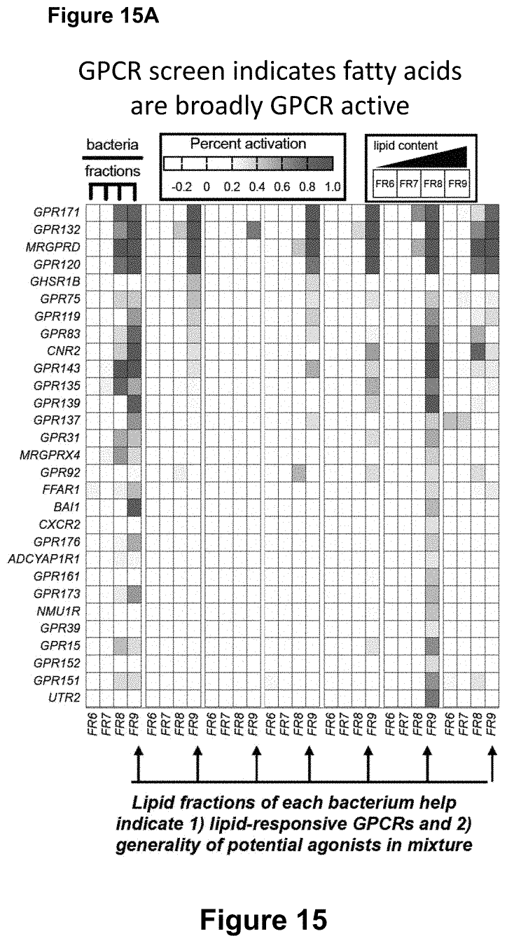

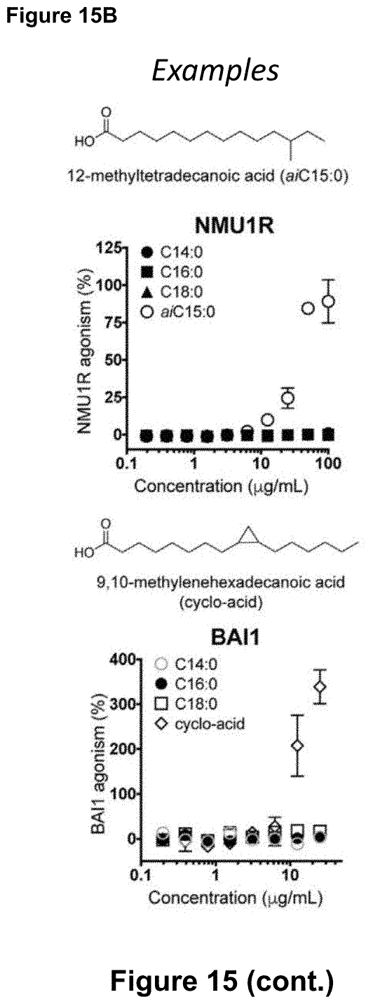

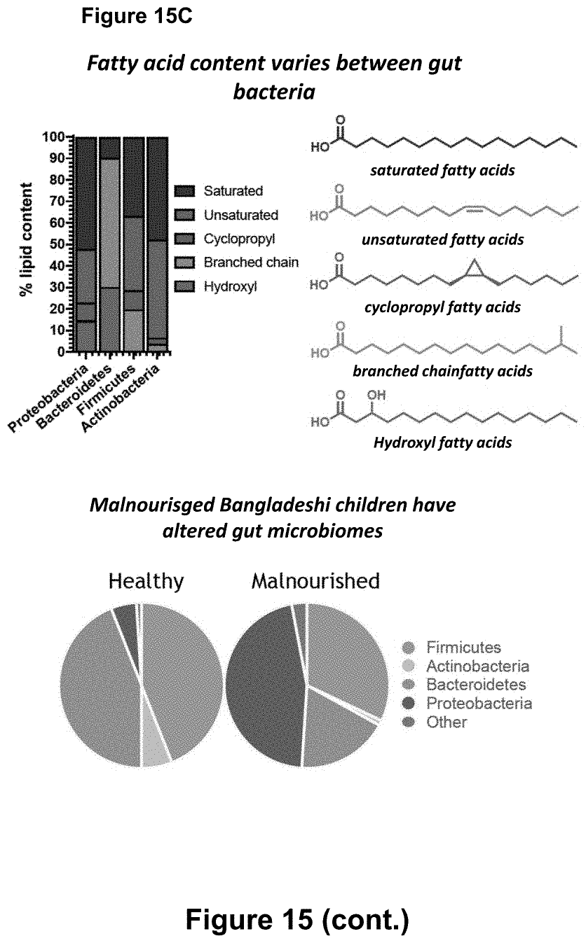

In one embodiment, the metabolites of the invention are derived from a desired microorganism and the metabolite binds to a receptor including but not limited to the receptors listed in FIG. 2B, FIG. 15, and Table 1. In various embodiments, the receptor is a G-protein coupled receptor (GPCR), nuclear receptors (NR), or a combination thereof.

In various embodiments, the microbial metabolite is a polypeptide, a carbohydrate, an oligosaccharide, a polysaccharide, a polynucleotide, a lipid, a phospholipid, a fatty acid, a steroid, a peptide, an amino acid, or any combination thereof. In various embodiments, the microbial metabolite is tyramine, tryptamine, cadaverine, 9,10-methylenehexadecanoic acid, 12-methyltetradecanoic acid (also referred to as 12-methylmyristic acid), 13-methyltetradecanoic acid (also referred to as 13-methylmyristic acid), 14-methylpalmitic acid, 4-hydroxycinnamic acid, anteiso-fatty acid, iso-fatty acid, or any combination thereof.

For example, in one embodiment, tyramine binds to DRD1, DRD2L, DRD2S, DRD3, DRD4, DRDS, or any combination thereof. In one embodiment, tryptamine binds to HTR1A, HTR1B, HTR1F, HTR1E, HTR2A, HTR2C, HTRSA, or any combination thereof. In one embodiment, cadaverine binds to HRH1, HRH2, HRH3, HRH4, or any combination thereof In one embodiment, 9,10-methylenehexadecanoic acid binds to BAIL CNR2, UTR2, or any combination thereof. In one embodiment, 12-methyltetradecanic acid binds to NMU1R, GPR151, UTR2, ADCYAP1R1, or any combination thereof. In one embodiment, 13-methyltetradecanic acid binds to GPR151. In one embodiment, 14-methylpalmitic acid binds to GPR151. In one embodiment, 4-hydroxycinnamic acid binds to GPR109B. In one embodiment, anteiso-fatty acids bind to NMU1R, UTR2, GPR120, or any combination thereof. In one embodiment, iso-fatty acid bind NMU1R, UTR2, GPR120, or any combination thereof.

In one aspect of the invention, the therapeutic composition is used to prevent or treat a disease or disorder in a subject in need thereof. In various embodiments, the disease or disorder is associated with dysfunction of at least one receptor listed in FIG. 2B, FIG. 15, and Table 1. In some embodiments, the disease or disorder is associated with the level (e.g., activity, expression, level, etc.) of at least one microorganism and/or a microbial metabolite.

### Methods of Screening Microbial Metabolites

The present invention relates to methods of screening and identifying microbial metabolite test compounds to identify microbial metabolite compounds that bind to a host receptor. In one embodiment, the invention comprises assessing whether the microbial metabolite test compound binds to a host receptor. In another embodiment, the invention comprises assessing whether the microbial metabolite test compound is an agonist of a host receptor. In another embodiment, the invention comprises assessing whether the microbial metabolite test compound is an antagonist of a host receptor. In some embodiments, the host receptor is a known host receptor. In other embodiments, the host receptor is an unknown host receptor.

In various embodiments, the host receptors that can interact with a microbial metabolite are GPCR and/or NR. In one embodiment, the NR activity is reduced. In one embodiment, the NR activity is increased. In one embodiment, the GPCR activity is reduced. In one embodiment, the GPCR activity is increased. Non-limiting examples of GPCR include

ADCYAP1R1, ADORA3, ADRA1B, ADRA2A, ADRA2B, ADRA2C, ADRBI, ADRB2, AGTRI, AGTRLI, AVPR1A, AVPR1B, AVPR2, BAI1, BAI2, BAI3, BDKRB1, BDKRB2, BRS3, C3ARI, CSARI, C5L2, CALCR, CALCRL-RAMPI, CALCRL-RAMP2, CALCRL-RAMP3, CALCR-RAMP2, CALCR-RAMP3, CCKAR, CCKBR, CCR1, CCR10, CCR2, CCR3, CCR4, CCRS, CCR6, CCR7, CCR8, CCR9, CCRL2, CHRM1, CHRM2, CHRM3, CHRM4, CHRMS, CMKLRI, CNR1, CNR2, CRHR1, CRHR2, CRTH2, CX3CR1, CXCR1, CXCR2, CXCR3, CXCR4, CXCRS, CXCR6, CXCR7, DARC, DRD1, DRD2L, DRD2S, DRD3, DRD4, DRDS, EBI2, EDG1, EDG3, EDG4, EDGS, EDGE, EDG7, EDNRA, EDNRB, F2R, F2RL1, F2RL3, FFARI, FPRI, FPRLI, FSHR, G2A, GALR1, GALR2, GCGR, GHSR, GHSRIB, GIPR, GLP1R, GLP2R, GPR1, GPR101, GPR103, GPR107, GPR109A, GPR109B, GPR117, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37LI, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTRI, HCRTR2, HRH1, HRH2, HRH3, HRH4, HTR1A, HTR1B, HTR1E, HTR1F, HTR2A, HTR2C, HTRSA, KISSIR, LGR4, LGRS, LGR6, LHCGR, LTB4R, MC1R, MC3R, MC4R, MCSR, MCHR1, MCHR2, MLNR, MRGPRD, MRGPRE, MRGPRF, MRGPRX1, MRGPRX2, MRGPRX4, MTNRIA, NMBR, NMU1R, NPBWR1, NPBWR2, NPFFR1, NPSRIB, NPY1R, NPY2R, NTSR1, OPNS, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, and VIPR2.

In various embodiments of the methods of the invention, the level (e.g., activity, expression, concentration, level, etc.) of GPCR is determined to be decreased or reduced when the relevant biomarkers (e.g., the activity of GPCR, expression of GPCR, concentration of GPCR, level of GPCR, etc.) are differentially expressed as compared to a comparator. In one embodiment, the microbial metabolite test compound is identified as an antagonist of GPCR when the level (e.g., activity, expression, concentration, level, etc.) of GPCR is decreased or reduced in the biological sample as compared to a comparator.

In various embodiments of the methods of the invention, the level (e.g., activity, expression, concentration, level, etc.) of GPCR is determined to be decreased or reduced when the level of GPCR (e.g., activity, expression, concentration, level, etc.) in the biological sample is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of GPCR is determined to be decreased or reduced when the level (e.g., activity, expression, concentration, level, etc.) of GPCR in the biological sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the microbial metabolite test compound is an antagonist of GPCR when the level (e.g., activity, expression, concentration, level, etc.) of GPCR is decreased or reduced in the biological sample as compared to a comparator. For example, in one embodiment, the microbial metabolite test compound is an antagonist of GPCR when the level (e.g., activity, expression, concentration, level, etc.) of GPCR is decreased by at least 1 fold, at least 1.1 fold, at least 1.2 fold, at least 1.3 fold, at least 1.4 fold, or at least 1.5 fold.

In various embodiments of the methods of the invention, the level (e.g., activity, expression, concentration, level, etc.) of NR is determined to be decreased or reduced when the relevant biomarkers (e.g., the activity of NR, expression of NR, concentration of NR, level of NR, etc.) are differentially expressed as compared to a comparator. In one embodiment, the microbial metabolite test compound is identified as an antagonist of NR when the level (e.g., activity, expression, concentration, level, etc.) of NR is decreased or reduced in the biological sample as compared to a comparator.

In various embodiments of the methods of the invention, the level (e.g., activity, expression, concentration, level, etc.) of NR is determined to be decreased or reduced when the level of NR (e.g., activity, expression, concentration, level, etc.) in the biological sample is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of NR is determined to be decreased or reduced when the level (e.g., activity, expression, concentration, level, etc.) of NR in the biological sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the microbial metabolite test compound is an antagonist of NR when the level (e.g., activity, expression, concentration, level, etc.) of NR is decreased or reduced in the biological sample as compared to a comparator. For example, in one embodiment, the microbial metabolite test compound is an antagonist of NR when the level (e.g., activity, expression, concentration, level, etc.) of NR is decreased by at least 1 fold, at least 1.1 fold, at least 1.2 fold, at least 1.3 fold, at least 1.4 fold, or at least 1.5 fold.

In various embodiments of the methods of the invention, the level (e.g., activity, expression, concentration, level, etc.) of GPCR is determined to be increased when the relevant biomarkers (e.g., the activity of GPCR, expression of GPCR, concentration of GPCR, level of GPCR, etc.) are differentially expressed as compared to a comparator. In one embodiment, the microbial metabolite test compound is identified as an agonist of GPCR when the level (e.g., activity, expression, concentration, level, etc.) of GPCR is increased in the biological sample as compared to a comparator.

In various embodiments of the methods of the invention, the level (e.g., activity, expression, concentration, level, etc.) of GPCR is determined to be increased when the level of GPCR (e.g., activity, expression, concentration, level, etc.) in the biological sample is increased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of GPCR is determined to be increased when the level (e.g., activity, expression, concentration, level, etc.) of GPCR in the biological sample is determined to be increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the microbial metabolite test compound is an agonist of GPCR when the level (e.g., activity, expression, concentration, level, etc.) of GPCR is increased in the biological sample as compared to a comparator. For example, in one embodiment, the microbial metabolite test compound is an agonist of GPCR when the level (e.g., activity, expression, concentration, level, etc.) of GPCR is increased by at least 1 fold, at least 1.1 fold, at least 1.2 fold, at least 1.3 fold, at least 1.4 fold, or at least 1.5 fold.

In various embodiments of the methods of the invention, the level (e.g., activity, expression, concentration, level, etc.) of NR is determined to be increased when the relevant biomarkers (e.g., the activity of NR, expression of NR, concentration of NR, level of NR, etc.) are differentially expressed as compared to a comparator. In one embodiment, the microbial metabolite test compound is identified as an agonist of NR when the level (e.g., activity, expression, concentration, level, etc.) of NR is increased in the biological sample as compared to a comparator.

In various embodiments of the methods of the invention, the level (e.g., activity, expression, concentration, level, etc.) of NR is determined to be increased when the level of NR (e.g., activity, expression, concentration, level, etc.) in the biological sample is increased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of NR is determined to be increased when the level (e.g., activity, expression, concentration, level, etc.) of NR in the biological sample is determined to be increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the microbial metabolite test compound is an agonist of NR when the level (e.g., activity, expression, concentration, level, etc.) of NR is increased in the biological sample as compared to a comparator. For example, in one embodiment, the microbial metabolite test compound is an agonist of NR when the level (e.g., activity, expression, concentration, level, etc.) of NR is increased by at least 1 fold, at least 1.1 fold, at least 1.2 fold, at least 1.3 fold, at least 1.4 fold, or at least 1.5 fold.

In one embodiment, the method comprises using a multi-dimensional non-linear algorithm to determine if the level (e.g., activity, expression, concentration, level, etc.) of a set of biomarkers in the biological sample is statistically different than the level in a comparator sample. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In one embodiment, the method comprises detecting one or more markers in a biological sample of the subject. In some embodiments, the level of one or more of markers of the invention in the biological test sample of the subject is compared with the level of the biomarker in a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level of the one or more biomarker in a sample obtained from a subject not having a disease or disorder, such as Crohn's disease. In one embodiment, the comparator is a level of the one or more biomarker in a sample obtained from a subject known not to have a disease or disorder, such as Crohn's disease.

In some the methods of the invention, a biological sample from a subject is assessed for the level of one or more of the markers of the invention in the biological sample obtained from the patient. The level of one or more of the markers of the invention in the biological sample can be determined by assessing the amount of polypeptide of one or more of the biomarkers of the invention in the biological sample, the amount of mRNA of one or more of the biomarkers of the invention in the biological sample, the amount of enzymatic activity of one or more of the biomarkers of the invention in the biological sample, or any combination thereof.

In certain embodiments, the biological sample obtained from the subject comprises gastrointestinal tissue of the subject, including gastrointestinal tissue excised during biopsy. In certain embodiments, the method comprises examining relevant biomarkers and their expression level. In one embodiment, biomarker expression includes transcription into messenger RNA (mRNA) and translation into protein. In one embodiment, the biomarker types comprise mRNA biomarkers. In various embodiments, the mRNA is detected by at least one of mass spectroscopy, PCR microarray, thermal sequencing, capillary array sequencing, solid phase sequencing, and the like. In another embodiment, the biomarker types comprise polypeptide biomarkers. In various embodiments, the polypeptide is detected by at least one of ELISA, Western blot, flow cytometry, immunofluorescence, immunohistochemistry, mass spectroscopy, and the like.

Biological samples may be of any biological tissue or fluid. Frequently the sample will be a “clinical sample” which is a sample derived from a patient. The biological sample may contain any biological material suitable for detecting the desired biomarkers, and may comprise cellular and/or non-cellular material obtained from the individual. A biological sample can be obtained by appropriate methods, such as, by way of examples, blood draw, fluid draw, biopsy, or surgical resection. Examples of such samples include but are not limited to blood, lymph, urine, gastrointestinal fluid, semen, and biopsies. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, tissue sample obtained during surgical resection, and archival samples with known diagnosis, treatment and/or outcome history. In certain embodiments, the biological sample comprises gastrointestinal tissue. In certain embodiments, the biological sample comprises gastrointestinal tissue of a subject having gastrointestinal cancer.

In some embodiments, the methods of the invention use live cells to perform experiments as the basis for the identification of a microbial metabolite test compound as a compound that interacts with a host receptor.

Microbial metabolites test compounds are applied to the assay system in the methods of the invention and alterations in parameters are detected. Microbial metabolites test compounds useful in the methods of the invention include known microbial metabolites, and unknown microbial metabolites, as well as complex mixtures of microbial metabolites, such as those obtained from microbial culture supernatants or from microbial culture lysates. Microbial cultures can be grown under a variety of different conditions to alter the array of microbial metabolites present in the microbial culture supernatant or from microbial culture lysate.

In various embodiments, assay systems useful in the methods of the invention comprise live cells that are sensitive to the effect of particular known and unknown microbial metabolites. Non-limiting examples of live cells useful in the methods of the invention to identify microbial metabolites having activity by alteration of particular parameters include epithelial cells, myeloid cells, basophils, neutrophils, eosinophils, monocytes, macrophages, natural killer (NK) cells, macrophages, dendritic cells (DC), lymphocytes, innate lymphoid cells (ILC), B cells, and T cells. Measurable parameters that may be altered in response to the exposure of cells to a microbial metabolite include, but are not limited to, epithelial permeability, tight junction complex composition and expression, mucus production (e.g., expression of mucins, etc.), expression of inflammatory cytokines and chemokines (e.g., IL-1, IL-6, IL-12, IL-13, IL-17, IL-22, IL-23, IL-25, IL-33, TSLP, TNF, IFNg, etc.), expression of anti-inflammatory cytokines and chemokines (e.g., IL-10, TGF-b, etc.), stimulation of TLRs and other receptors (e.g., dectins, NODs, RLRs, etc.), maturation (e.g., expression of MHC-II, CD80/86, CD40, etc.), differentiation, ability to activate T cells, ability to differentiate T cells into effector subsets (TH1, TH2, TH9, TH17, Th22, Tregs, etc.), ability of innate lymphoid cells (ILC) to produce cytokines (e.g., IL-22, IL-17, IL-13, IFNg, etc.) when stimulated by IL-23, TSLP, IL-33, IL-25, etc., ability of B cells to produce antibodies of different isotypes (e.g., IgA, IgM, IgG, IgE, IgD, etc.), ability of T cells to become activated and differentiated into different effector lineages (TH1, TH2, TH9, TH17, Th22, Tregs, etc.) with, or without, stimulated by aCD3, aCD28, DCs, etc., or combinations thereof.

When microbial metabolites are detected to alter particular parameters, identification of the metabolite or metabolites responsible for the altered parameter is performed. Various methods are known in the art for identifying an unknown compound in a complex mixture. Individual components may be separated, analyzed and characterized using methods known to those skilled in the art. In a non-limiting embodiment, the individual components may be partially or completely purified using, for example, chromatographic methods (such as, but not limited to, high performance liquid chromatography (HPLC), silica gel chromatography or alumina chromatography), selective crystallization or precipitation, or selective solvent extraction. In another non-limiting embodiment, the partially or completely purified components of the library may be analyzed or characterized using methods such as, but not limited to, nuclear magnetic resonance (NMR), mass spectrometry (MS), liquid chromatography-mass spectrometry (LC-MS), ultraviolet-visible (UV-vis) spectroscopy, and infrared (IR) spectroscopy. The information derived from these methods may be used to establish the structure of the specific components of the library.

In some embodiments, the assays and methods comprise high content screening (HCS) of suitable microbial metabolite test compounds. In some embodiments, the assays and methods comprise high content screening (HCS) of suitable microbial metabolite test compounds. Typically, HCS is an automated system to enhance the throughput of the screening process. However, the present invention is not limited to the speed or automation of the screening process.

In some embodiments, the methods of the invention provide for screening of a single microbial metabolite test compound. In other embodiments, the methods of the invention provide for screening of thousands of microbial metabolite test compounds. In another aspect of the invention, the invention relates to selection of a microbial metabolite from a library of microbial metabolite test compounds. Compounds tested in the screening methods of the present invention are not limited to a specific type of microbial metabolite test compound. Non-limiting examples of potential microbial metabolite test compounds include chemical agents, metabolites, pharmaceuticals, peptides, proteins (such as antibodies, cytokines, enzymes, etc.), fatty acids, nucleic acids, tyramine, tryptamine, cadaverine, 9,10-methylenehexadecanoic acid, 12-methyltetradecanoic acid (also referred to as 12-methylmyristic acid), 13-methyltetradecanoic acid (also referred to as 13-methylmyristic acid), 14-methylpalmitic acid, 4-hydroxycinnamic acid, anteiso-fatty acid, and iso-fatty acid.

In one embodiment, entire microbial metabolite test compound libraries are screened. Microbial metabolite test compound libraries are a large collection of stored compounds utilized for screening, including high-throughput screening. Microbial metabolite test compounds in a library may have no relation to one another, or alternatively have a common characteristic. For example, a hypothetical microbial metabolite test compound library may contain all known compounds known to bind to a specific type of host receptor. As would be understood by one skilled in the art, the methods of the invention are not limited to the types of compound libraries screened. A non-limiting example of a compound library includes the set from CGMCC (see english.im.cas.cn/rh/rd/brc/).

In one embodiment, the methods of the invention relate to high throughput screening methods and automated screening of large quantities of microbial metabolite test compounds to identify specific metabolites that interact with a host receptor.

In one embodiment, the assay of the invention may also be used to test delivery vehicles. These may be of any form, from conventional pharmaceutical formulations, to gene delivery vehicles. For example, the assay may be used to compare the effects of the same compound administered by two or more different delivery systems (e.g. a depot formulation and a controlled release formulation). Thus, the microbial metabolite test compound may be delivered by a delivery vehicle of any appropriate type with or without any associated therapeutic agent.

In one embodiment, compounds are evaluated alone. In another embodiment, compounds are evaluated when delivered along with a delivery vehicle. Non-limiting examples of delivery vehicles include polymersomes, vesicles, micelles, plasmid vectors, viral vectors, and the like. As described elsewhere herein, microbial metabolite test compounds are evaluated for their ability to interact with a host receptor. In one embodiment, the methods of the invention comprise selecting a microbial metabolite test compound that is an agonist of a host receptor. In another embodiment, the methods of the invention comprise selecting a microbial metabolite test compound that is an antagonist of a host receptor. In another embodiment, microbial metabolite test compound are delivered along with another known agents to determine whether the microbial metabolite test compounds exhibit interference or synergy with other agents.

The microbial metabolite test compound may be added to the assay method to be tested by any suitable means. For example, the microbial metabolite test compound may be injected into the cells of the assay, or it can be added to the nutrient medium and allowed to diffuse into the cells.

In one embodiment, the screening methods involve providing a library containing a large number of microbial metabolite test compounds, at least one of which potentially having an activity through its interaction with a host receptor. Such a library is then screened in one or more assays, as described herein, to identify those library members (particular chemical species or subclasses) that display a desired characteristic activity. The compounds thus identified can serve as conventional “hit compounds” or can themselves be used as potential or actual therapeutics. It is typical to that new chemical entities with useful properties are generated by identifying a chemical compound (called a “hit compound”) with some desirable property or activity, and evaluating the property of those compounds. The invention includes such hit compounds, as well as compounds derived from such hit compounds.

Thus, the present invention also relates to methods of screening and identifying drug test compounds to identify drug compounds that bind to a host receptor. In one embodiment, the invention comprises assessing whether the drug test compound binds to a host receptor. In another embodiment, the invention comprises assessing whether the drug test compound is an agonist of a host receptor. In another embodiment, the invention comprises assessing whether the drug test compound is an antagonist of a host receptor. In some embodiments, the host receptor is a known host receptor. In various embodiments, the host receptors that can interact with a drug compound are GPCR and/or NR. In one embodiment, the NR activity is reduced. In one embodiment, the NR activity is increased. In one embodiment, the GPCR activity is reduced. In one embodiment, the GPCR activity is increased. In other embodiments, the host receptor is an unknown host receptor.

In various embodiments, the methods of screening and identifying drug test compounds to identify drug compounds that bind to a host receptor are any of the methods described herein.

### Methods of Screening Host Receptors

The present invention relates to methods of screening and identifying host receptors by their ability to interact with a microbial metabolite. Non-limiting examples of host receptors that can interact with a microbial metabolite include GPCR and NR.

In one embodiment, the methods of the invention include cells that express a candidate host receptor. Candidate host receptors can be identified based upon their expression in cell types that are known to interact with microbial metabolites. Non-limiting examples of cell types that express receptors that may interact with microbial metabolites include cells of the immune system, such as those in tissues that are colonized by microbes, including the gastrointestinal tract, the respiratory tract, the urogenital tract, and skin. Non-limiting examples of immune system host cells that may express candidate host receptors include epithelial cells, myeloid cells, basophils, neutrophils, eosinophils, monocytes, macrophages, dendritic cells (DC), lymphocytes, natural killer cells, etc. Optionally, bioinformatic analysis is performed to identify sequence similarity of candidate host receptors with receptors with known specificities to microbial metabolites. Although not necessary for the practice of the methods of the invention described herein, the identification of sequence similarity of candidate host receptors with known specificities can aid in the identification of a chemical class of potential ligands for the candidate host receptor, including, for example, fatty acids, biogenic amines, carboxylic acids, etc.

In various embodiments, candidate receptors are expressed in cells designed to report on microbial metabolite binding to the candidate receptors. In various embodiments, the candidate host receptor is tagged with a selectable marker, such as an antibiotic resistance gene, or a detectable marker, such as a fluorescent tag. Non-limiting examples of fluorescent tags include green fluorescent protein (GFP), cyan fluorescent protein (CFP), yellow fluorescent protein (YFP), red fluorescent protein (RFP), orange fluorescent protein (OFP), eGFP, mCherry, hrGFP, hrGFPII, Alexa 488, Alexa 594, and the like. Fluorescent tags may also be photoconvertible, such as for example kindling red fluorescent protein (KFP-red), PS-CFP2, Dendra2, CoralHue Kaede and CoralHue Kikume. However, the invention should not be limited to a particular label. Rather, any detectable label can be used to tag the candidate host receptor.

There are a variety of means and protocols for the expression of exogenous proteins in cells including, but not limited to, transformation, transfection, cell or protoplast fusion, use of a chemical treatment (e.g., polyethylene glycol treatment of protoplasts, calcium treatment, transfecting agents such as LIPOFECTINTM and LIPOFECTAMINETM transfection reagents available from Invitrogen (Carlsbad, Calif.), use of various types of liposomes, use of a mechanical device (e.g., nucleic acid coated microbeads), use of electrical charge (e.g., electroporation), and combinations thereof. It is within the skill of a practitioner in the art to determine the particular protocol and/or means to use to insert a particular vector molecule described herein into a desired host cell.

Employing genetic engineering technology necessarily requires growing recombinant host cells (e.g., transfectants, transformants) under a variety of specified conditions as determined by the requirements of the cells and the particular cellular state desired by the practitioner. In one embodiment, genetic engineering includes transiently transfected cells or the establishment of stable expression cell lines. For example, a host cell may possess (as determined by its genetic disposition) certain nutritional requirements, or a particular resistance or sensitivity to physical (e.g., temperature) and/or chemical (e.g., antibiotic) conditions. In addition, specific culture conditions may be necessary to regulate the expression of a desired gene (e.g., the use of inducible promoters). These varied conditions and the requirements to satisfy such conditions are understood and appreciated by practitioners in the art.

The recombinant vectors harboring the sequence encoding the candidate host receptor tagged with a marker, or other elements of the present invention, can be introduced into an appropriate host cell by any means known in the art. For example, the vector can be transfected into the host cell by calcium phosphate co-precipitation, by conventional mechanical procedures such as microinjection or electroporation, by insertion of a plasmid encased in liposomes, and by virus vectors. These techniques are all well-known and routinely practiced in the art, e.g., Brent et al., Current Protocols in Molecular Biology, John Wiley & Sons, Inc. (2012). Host cells which harbor the transfected recombinant vector can be identified and isolated using the selection marker present on the vector. Large numbers of recipient cells may then be grown in a medium which selects for vector-containing cells. These cells may be used directly or the expressed recombinant protein may be purified in accordance with conventional methods such as extraction, precipitation, chromatography, affinity methods, electrophoresis and the like. The exact procedure used will depend upon the specific protein produced and the specific vector/host expression system utilized.

In an embodiment, host cells for expressing the recombinant vectors are eukaryotic cells. Eukaryotic vector/host systems, and mammalian expression systems, allow for proper post-translational modifications of expressed mammalian proteins to occur, e.g., proper processing of the primary transcript, glycosylation, phosphorylation and advantageously secretion of expressed product. Therefore, eukaryotic cells such as mammalian cells can be the host cells for the host receptor of interest. Examples of such host cells include primary cells and cell lines, such as CHO, BHK, HEK293, VERO, HeLa, COS, MDCK, NSO and W138. Such cells lines can be transiently transfected with a candidate host receptor and/or other elements of the invention. Alternatively, stable cell lines genetically altered to constitutively express a candidate host receptor and/or other elements of the invention can be generated by methods known in the art.

In some embodiments, engineered host cell systems that utilize recombinant viruses or viral elements to direct expression of the receptor of interest are employed. For example, when using adenovirus expression vectors, the coding sequence of the receptor of interest may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the polypeptide of interest in infected hosts (e.g., see Logan & Shenk, 1984Proc. Natl. Acad. Sci. USA 81:3655-3659). Alternatively, the vaccinia virus 7.5K promoter may be used. (e.g., see, Mackett et al., 1982, Proc. Natl. Acad. Sci. USA, 79:7415-7419; Mackett et al., 1984, J. Virol. 49:857-864; Panicali et al., 1982, Proc. Natl. Acad. Sci. USA, 79:4927-4931). Of particular interest are vectors based on bovine papilloma virus which have the ability to replicate as extrachromasomal elements (Sarver et al., 1981, Mol. Cell. Biol. 1:486). These vectors can be used for stable expression by including a selectable marker in the plasmid, such as an antibiotic resistance gene, such as the neo gene. Alternatively, the retroviral genome can be modified for use as a vector capable of introducing and directing the expression of the gene of interest in host cells (Cone & Mulligan, 1984, Proc. Natl. Acad. Sci. USA 8 1:6349-6353). High level expression may also be achieved using inducible promoters, including, but not limited to, the metallothionine IIA promoter and heat shock promoters.

The assays and methods of the invention comprise the steps of applying a test microbial metabolite to a cell expression a candidate host receptor to identify ligands for the candidate receptor. Microbial metabolites useful in the methods of the invention include known microbial metabolites, and unknown microbial metabolites, as well as complex mixtures of microbial metabolites, such as those obtained from microbial culture supernatants or from microbial culture lysates. Microbial cultures can be grown under a variety of different conditions to alter the array of microbial metabolites present in the microbial culture supernatant or in the microbial culture lysate.

The microbial metabolite test compound may be added to the assay method to be tested by any suitable means. For example, cells can be contacted with the microbial metabolite test compound to allow activation of cellular receptors either on the cell surface (e.g., GPCRs) or inside the cells (e.g., NRs).

When cells expressing a candidate host receptor exhibits activity in response to an unknown microbial metabolite, identification of the metabolite or metabolites responsible is performed. Various methods are known in the art for identifying an unknown compound in a complex mixture. Individual components may be separated, analyzed and characterized using methods known to those skilled in the art. In a non-limiting embodiment, the individual components may be partially or completely purified using, for example, chromatographic methods (such as, but not limited to, high performance liquid chromatography (HPLC), silica gel chromatography or alumina chromatography), selective crystallization or precipitation, or selective solvent extraction. In another non-limiting embodiment, the partially or completely purified components of the library may be analyzed or characterized using methods such as, but not limited to, nuclear magnetic resonance (NMR), mass spectrometry (MS), liquid chromatography-mass spectrometry (LC-MS), ultraviolet-visible (UV-vis) spectroscopy, and infrared (IR) spectroscopy. The information derived from these methods may be used to establish the structure of the specific components of the library.

When a cell expressing a candidate host receptor exhibits activity in response to a microbial metabolite, additional compound screens, including compounds that are not microbial metabolites, can be performed to identify additional ligands, including agonists and antagonists, of the receptor.

The host receptor thus identified can serve as conventional “hit compound” or can themselves be used as a target of potential or actual therapeutics. It is typical to that new chemical entities with useful properties are generated by identifying a chemical compound (called a “hit compound”) with some desirable property or activity, and evaluating the property of those compounds. The invention includes such hit compounds, as well as compounds derived from such hit compounds.

Thus, the present invention also relates to methods of screening and identifying drug compounds by their ability to interact with a microbial metabolite. In one embodiment, the methods of the invention include cells that express a candidate drug compounds. Candidate drug compounds can be identified based upon their expression in cell types that are known to interact with microbial metabolites. In various embodiments, candidate drug compounds are expressed in cells designed to report on microbial metabolite binding to the drug compounds.

In various embodiments, the methods of screening and identifying drug test compounds by their ability to interact with a microbial metabolite are any of the methods described herein.

In one embodiment, the drug test compound is identified to interact with a microbial metabolite when the level (e.g., activity, expression, concentration, level, etc.) of microbial metabolite is decreased or reduced in the biological sample as compared to a comparator. In various embodiments of the methods of the invention, the level (e.g., activity, expression, concentration, level, etc.) of microbial metabolite is determined to be decreased or reduced when the relevant biomarkers (e.g., the activity of microbial metabolite, expression of microbial metabolite, concentration of microbial metabolite, level of microbial metabolite, etc.) are differentially expressed as compared to a comparator.

In various embodiments of the methods of the invention, the level (e.g., activity, expression, concentration, level, etc.) of microbial metabolite is determined to be decreased or reduced when the level of microbial metabolite (e.g., activity, expression, concentration, level, etc.) in the biological sample is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of microbial metabolite is determined to be decreased or reduced when the level (e.g., activity, expression, concentration, level, etc.) of microbial metabolite in the biological sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the drug test compound is identified to interact with a microbial metabolite when the level (e.g., activity, expression, concentration, level, etc.) of microbial metabolite is decreased or reduced in the biological sample as compared to a comparator. For example, in one embodiment, the drug test compound is identified to interact with a microbial metabolite when the level (e.g., activity, expression, concentration, level, etc.) of microbial metabolite is decreased by at least 1 fold, at least 1.1 fold, at least 1.2 fold, at least 1.3 fold, at least 1.4 fold, or at least 1.5 fold.

In one embodiment, the drug test compound is identified to interact with a microbial metabolite when the level (e.g., activity, expression, concentration, level, etc.) of microbial metabolite is increased in the biological sample as compared to a comparator. In various embodiments of the methods of the invention, the level (e.g., activity, expression, concentration, level, etc.) of microbial metabolite is determined to be increased when the relevant biomarkers (e.g., the activity of microbial metabolite, expression of microbial metabolite, concentration of microbial metabolite, level of microbial metabolite, etc.) are differentially expressed as compared to a comparator.

In various embodiments of the methods of the invention, the level (e.g., activity, expression, concentration, level, etc.) of microbial metabolite is determined to be increased when the level of microbial metabolite (e.g., activity, expression, concentration, level, etc.) in the biological sample is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of microbial metabolite is determined to be increased when the level (e.g., activity, expression, concentration, level, etc.) of microbial metabolite in the biological sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, the drug test compound is identified to interact with a microbial metabolite when the level (e.g., activity, expression, concentration, level, etc.) of microbial metabolite is increased in the biological sample as compared to a comparator. For example, in one embodiment, the drug test compound is identified to interact with a microbial metabolite when the level (e.g., activity, expression, concentration, level, etc.) of microbial metabolite is decreased by at least 1 fold, at least 1.1 fold, at least 1.2 fold, at least 1.3 fold, at least 1.4 fold, or at least 1.5 fold.

### Therapy and Methods of Treating or Preventing Diseases or Disorders

The present invention also relates, in part, to a method of treating a disease or disorder. In various embodiments, the methods of invention comprise administering any composition described herein to a subject in need thereof. In various embodiments, the disease or disorder is associated with the level (e.g., activity, expression, level, etc.) of at least one receptor listed in FIG. 2B, FIG. 15, and Table 1. In various embodiments, the disease or disorder is associated with dysfunction of at least one receptor listed in FIG. 2B, FIG. 15, and Table 1. In some embodiments, the disease or disorder is associated with the level (e.g., activity, expression, level, etc.) of at least one microorganism and/or a microbial metabolite.

In various embodiments, the disease or disorder is associated with at least one receptor listed in FIG. 2B, FIG. 15, and Table 1. In one embodiment, the disease or disorder is associated with GPCR, NR, or a combination theref In some embodiments, the disease or disorder is associated with GPCR is a disease or disorder is associated with ADCYAP1R1, ADORA3, ADRA1B, ADRA2A, ADRA2B, ADRA2C, ADRBI, ADRB2, AGTRI, AGTRLI, AVPR1A, AVPR1B, AVPR2, BAIL BAI2, BAI3, BDKRB1, BDKRB2, BRS3, C3ARI, CSARI, C5L2, CALCR, CALCRL-RAMPI, CALCRL-RAIVIP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLRI, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRD5, EBI2, EDG1, EDG3, EDG4, EDG5, EDGE, EDG7, EDNRA, EDNRB, F2R, F2RL1, F2RL3, FFARI, FPRI, FPRLI, FSHR, G2A, GALR1, GALR2, GCGR, GHSR, GHSRIB, GIPR, GLP1R, GLP2R, GPR1, GPR101, GPR103, GPR107, GPR109A, GPR109B, GPR117, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37LI, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTRI, HCRTR2, HRH1, HRH2, HRH3, HRH4, HTR1A, HTR1B, HTR1E, HTR1F, HTR2A, HTR2C, HTRSA, KISSIR, LGR4, LGRS, LGR6, LHCGR, LTB4R, MC1R, MC3R, MC4R, MCSR, MCHR1, MCHR2, MLNR, MRGPRD, MRGPRE, MRGPRF, MRGPRX1, MRGPRX2, MRGPRX4, MTNRIA, NMBR, NMU1R, NPBWR1, NPBWR2, NPFFR1, NPSRIB, NPY1R, NPY2R, NTSR1, OPNS, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTRS, TAARS, TACR1, TACR2, TACR3, TBXA2R, TRHR, TSHR(L), UTR2, VIPR1, VIPR2, or any combination thereof.

In various embodiments, the disease or disorder is associated with an increased level (e.g., activity, expression, level, etc.) of at least one receptor listed in FIG. 2B, FIG. 15, and Table 1. In one embodiment, the disease or disorder is associated with an increased level (e.g., activity, expression, level, etc.) of at least one GPCR. In one embodiment, the disease or disorder is associated with an increased level (e.g., activity, expression, level, etc.) of at least one NR.

In one embodiment, the disease or disorder is associated with an increased level of GPCR when the level (e.g., activity, expression, concentration, level, etc.) of GPCR is increased in the biological sample as compared to a comparator. In various embodiments of the methods of the invention, the level (e.g., activity, expression, concentration, level, etc.) of GPCR is determined to be increased when the relevant biomarkers (e.g., the activity of GPCR, expression of GPCR, concentration of GPCR, level of GPCR, etc.) are differentially expressed as compared to a comparator. In certain embodiments, the comparator may be at the level of the relevant biomarkers (e.g., the activity of GPCR, expression of GPCR, concentration of GPCR, level of GPCR, etc.) in a subject not having a disease or disorder associated with increased level (e.g., activity, expression, concentration, level, etc.) of GPCR, a subject not at risk of developing a disease or disorder associated with increased level (e.g., activity, expression, concentration, level, etc.) of GPCR, a population not having a disease or disorder associated with increased level (e.g., activity, expression, concentration, level, etc.) of GPCR, or a population not having a risk of developing a disease or disorder associated with increased level (e.g., activity, expression, concentration, level, etc.) of GPCR.

In one embodiment, the disease or disorder is associated with an increased level of NR when the level (e.g., activity, expression, concentration, level, etc.) of NR is increased in the biological sample as compared to a comparator. In various embodiments of the methods of the invention, the level (e.g., activity, expression, concentration, level, etc.) of NR is determined to be increased when the relevant biomarkers (e.g., the activity of NR, expression of NR, concentration of NR, level of NR, etc.) are differentially expressed as compared to a comparator. In certain embodiments, the comparator may be at the level of the relevant biomarkers (e.g., the activity of NR, expression of NR, concentration of NR, level of NR, etc.) in a subject not having a disease or disorder associated with increased level (e.g., activity, expression, concentration, level, etc.) of NR, a subject not at risk of developing a disease or disorder associated with increased level (e.g., activity, expression, concentration, level, etc.) of NR, a population not having a disease or disorder associated with increased level (e.g., activity, expression, concentration, level, etc.) of NR, or a population not having a risk of developing a disease or disorder associated with increased level (e.g., activity, expression, concentration, level, etc.) of NR.

In various embodiments, the disease or disorder is associated with a decreased or reduced level (e.g., activity, expression, level, etc.) of at least one receptor listed in FIG. 2B, FIG. 15, and Table 1. In one embodiment, the disease or disorder is associated with a decreased or reduced level (e.g., activity, expression, level, etc.) of at least one GPCR. In one embodiment, the disease or disorder is associated with a decreased or reduced level (e.g., activity, expression, level, etc.) of at least one NR.

In one embodiment, the disease or disorder associated with a decreased or reduced level of GPCR when the level (e.g., activity, expression, concentration, level, etc.) of GPCR is decreased in the biological sample as compared to a comparator. In various embodiments of the methods of the invention, the level (e.g., activity, expression, concentration, level, etc.) of GPCR is determined to be decreased when the relevant biomarkers (e.g., the activity of GPCR, expression of GPCR, concentration of GPCR, level of GPCR, etc.) are differentially expressed as compared to a comparator. In certain embodiments, the comparator may be at the level of the relevant biomarkers (e.g., the activity of GPCR, expression of GPCR, concentration of GPCR, level of GPCR, etc.) in a subject not having a disease or disorder associated with decreased level (e.g., activity, expression, concentration, level, etc.) of GPCR, a subject not at risk of developing a disease or disorder associated with decreased level (e.g., activity, expression, concentration, level, etc.) of GPCR, a population not having a disease or disorder associated with decreased level (e.g., activity, expression, concentration, level, etc.) of GPCR, or a population not having a risk of developing a disease or disorder associated with decreased level (e.g., activity, expression, concentration, level, etc.) of GPCR.

In one embodiment, the disease or disorder associated with a decreased or reduced level of NR when the level (e.g., activity, expression, concentration, level, etc.) of NR is decreased in the biological sample as compared to a comparator. In various embodiments of the methods of the invention, the level (e.g., activity, expression, concentration, level, etc.) of NR is determined to be decreased when the relevant biomarkers (e.g., the activity of NR, expression of NR, concentration of NR, level of NR, etc.) are differentially expressed as compared to a comparator. In certain embodiments, the comparator may be at the level of the relevant biomarkers (e.g., the activity of NR, expression of NR, concentration of NR, level of NR, etc.) in a subject not having a disease or disorder associated with decreased level (e.g., activity, expression, concentration, level, etc.) of NR, a subject not at risk of developing a disease or disorder associated with decreased level (e.g., activity, expression, concentration, level, etc.) of NR, a population not having a disease or disorder associated with decreased level (e.g., activity, expression, concentration, level, etc.) of NR, or a population not having a risk of developing a disease or disorder associated with decreased level (e.g., activity, expression, concentration, level, etc.) of NR.

In some embodiments, the methods of the present invention modulate the level (e.g., activity, expression, level, etc.) of at least one receptor listed in FIG. 2B, FIG. 15, and Table 1. In some embodiments, the methods of the present invention modulate the level (e.g., activity, expression, level, etc.) of at least one GPCR. For example, in some embodiments, the methods of the present invention modulate the level (e.g., activity, expression, level, etc.) of ADCYAP1R1, ADORA3, ADRA1B, ADRA2A, ADRA2B, ADRA2C, ADRBI, ADRB2, AGTRI, AGTRLI, AVPR1A, AVPR1B, AVPR2, BAI1, BAI2, BAI3, BDKRB1, BDKRB2, BRS3, C3ARI, CSARI, C5L2, CALCR, CALCRL-RAMPI, CALCRL-RAMP2, CALCRL-RAMP3, CALCR-RAMP2, CALCR-RAMP3, CCKAR, CCKBR, CCR1, CCR10, CCR2, CCR3, CCR4, CCRS, CCR6, CCR7, CCR8, CCR9, CCRL2, CHRM1, CHRM2, CHRM3, CHRM4, CHRMS, CMKLRI, CNR1, CNR2, CRHR1, CRHR2, CRTH2, CX3CR1, CXCR1, CXCR2, CXCR3, CXCR4, CXCRS, CXCR6, CXCR7, DARC, DRD1, DRD2L, DRD2S, DRD3, DRD4, DRDS, EBI2, EDG1, EDG3, EDG4, EDGS, EDGE, EDG7, EDNRA, EDNRB, F2R, F2RL1, F2RL3, FFARI, FPRI, FPRLI, FSHR, G2A, GALR1, GALR2, GCGR, GHSR, GHSRIB, GIPR, GLP1R, GLP2R, GPR1, GPR101, GPR103, GPR107, GPR109A, GPR109B, GPR117, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37LI, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTRI, HCRTR2, HRH1, HRH2, HRH3, HRH4, HTR1A, HTR1B, HTR1E, HTR1F, HTR2A, HTR2C, HTRSA, KISSIR, LGR4, LGRS, LGR6, LHCGR, LTB4R, MC1R, MC3R, MC4R, MCSR, MCHR1, MCHR2, MLNR, MRGPRD, MRGPRE, MRGPRF, MRGPRX1, MRGPRX2, MRGPRX4, MTNRIA, NMBR, NMU1R, NPBWR1, NPBWR2, NPFFR1, NPSRIB, NPY1R, NPY2R, NTSR1, OPNS, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, VIPR2, or any combination thereof.

In some embodiments, the methods of the present invention reduce the level (e.g., activity, expression, level, etc.) of at least one receptor listed in FIG. 2B, FIG. 15, and Table 1. In some embodiments, the methods of the present invention reduce the level (e.g., activity, expression, level, etc.) of at least one GPCR or NR. For example, in one embodiment, the level (e.g., activity, expression, level, etc.) of GPCR is determined to be a decreased or reduced level of GPCR when the level (e.g., activity, expression, concentration, level, etc.) of GPCR is decreased by at least 1 fold, at least 1.1 fold, at least 1.2 fold, at least 1.3 fold, at least 1.4 fold, or at least 1.5 fold. In one embodiment, the level of NR is determined to be a decreased or reduced level of NR when the level (e.g., activity, expression, concentration, level, etc.) of NR is decreased by at least 1 fold, at least 1.1 fold, at least 1.2 fold, at least 1.3 fold, at least 1.4 fold, or at least 1.5 fold.

In some embodiments, the methods of the present invention increase the level (e.g., activity, expression, level, etc.) of at least one receptor listed in FIG. 2B, FIG. 15, and

Table 1. In some embodiments, the methods of the present invention increase the level (e.g., activity, expression, level, etc.) of at least one GPCR or NR. For example, in one embodiment, the level (e.g., activity, expression, level, etc.) of GPCR is determined to be an increased level of GPCR when the level (e.g., activity, expression, concentration, level, etc.) of GPCR is increased by at least 1 fold, at least 1.1 fold, at least 1.2 fold, at least 1.3 fold, at least 1.4 fold, or at least 1.5 fold. In one embodiment, the level of NR is determined to be an increased level of NR when the level (e.g., activity, expression, concentration, level, etc.) of NR is increased by at least 1 fold, at least 1.1 fold, at least 1.2 fold, at least 1.3 fold, at least 1.4 fold, or at least 1.5 fold.

In some aspects of the invention, the methods of the present invention modulate the level (e.g., activity, expression, level, etc.) of at least one receptor listed in FIG. 2B, FIG. 15 by administering a genetically engineered cell to a subject in need thereof. Thus, in various aspects of the invention, the methods of invention comprise administering a genetically engineered cell to a subject in need thereof. In some embodiments, the genetically engineered cell comprises a genetically engineered Escherichia coli LF82, genetically engineered Enterococcus faecalis, genetically engineered Lactobacilhs plantarum, genetically engineered Faecalibacterium prauznitzii, genetically engineered Bifidobacterium longum, genetically engineered Bacteroides vulgatus, genetically engineered Ruminococcus gnavus, or any combination thereof.

In some embodiments, the genetically engineered cell comprises a nucleic acid sequence set forth in SEQ ID NO: 1, or a variant or fragment thereof, nucleic acid sequence set forth in SEQ ID NO: 2, or a variant or a fragment thereof, nucleic acid sequence set forth in SEQ ID NO: 3, or a variant or fragment thereof, nucleic acid sequence set forth in SEQ ID NO: 4, or a variant or fragment thereof, nucleic acid sequence set forth in SEQ ID NO: 5, or a variant or fragment thereof, nucleic acid sequence set forth in SEQ ID NO: 6, or a variant or fragment thereof, nucleic acid sequence set forth in SEQ ID NO: 7, or a variant or fragment thereof, nucleic acid sequence set forth in SEQ ID NO: 8, or a variant or fragment thereof, nucleic acid sequence set forth in SEQ ID NO: 9, or a variant or fragment thereof, nucleic acid sequence set forth in SEQ ID NO: 10, or a variant or fragment thereof, nucleic acid sequence set forth in SEQ ID NO: 11, or a variant or fragment thereof, nucleic acid sequence set forth in SEQ ID NO: 12, or a variant or fragment thereof, nucleic acid sequence set forth in SEQ ID NO: 13, or a variant or fragment thereof, nucleic acid sequence set forth in SEQ ID NO: 14, or a variant or fragment thereof, or any combination thereof.

In one embodiment, the genetically engineered cell expresses a metabolite. In various embodiments, the metabolite binds to a receptor. In various embodiments, the receptor is at least one receptor listed in FIG. 2B, FIG. 15, and Table 1.

In some embodiments, the disease or disorder is associated with at least one microorganism and/or a microbial metabolite. In some embodiments, the disease or disorder is associated with an increased level (e.g., activity, expression, level, etc.) of at least one microorganism and/or a microbial metabolite.

In one embodiment, the disease or disorder is associated with an increased level of microbial metabolite when the level (e.g., activity, expression, concentration, level, etc.) of microbial metabolite is increased in the biological sample as compared to a comparator. In various embodiments of the methods of the invention, the level (e.g., activity, expression, concentration, level, etc.) of microbial metabolite is determined to be increased when the relevant biomarkers (e.g., the activity of microbial metabolite, expression of microbial metabolite, concentration of microbial metabolite, level of microbial metabolite, etc.) are differentially expressed as compared to a comparator. In certain embodiments, the comparator may be at the level of the relevant biomarkers (e.g., the activity of microbial metabolite, expression of microbial metabolite, concentration of microbial metabolite, level of microbial metabolite, etc.) in a subject not having a disease or disorder associated with increased level (e.g., activity, expression, concentration, level, etc.) of microbial metabolite, a subject not at risk of developing a disease or disorder associated with increased level (e.g., activity, expression, concentration, level, etc.) of microbial metabolite, a population not having a disease or disorder associated with increased level (e.g., activity, expression, concentration, level, etc.) of microbial metabolite, or a population not having a risk of developing a disease or disorder associated with increased level (e.g., activity, expression, concentration, level, etc.) of microbial metabolite.

In one embodiment, the disease or disorder is associated with an increased level of microorganism when the level (e.g., activity, expression, concentration, level, etc.) of microorganism is increased in the biological sample as compared to a comparator. In various embodiments of the methods of the invention, the level (e.g., activity, expression, concentration, level, etc.) of microorganism is determined to be increased when the relevant biomarkers (e.g., the activity of microorganism, expression of microorganism, concentration of microorganism, level of microorganism, etc.) are differentially expressed as compared to a comparator.

In various embodiments of the methods of the invention, the level (e.g., activity, expression, concentration, level, etc.) of microorganism is determined to be increased when the level of microorganism (e.g., activity, expression, concentration, level, etc.) in the biological sample is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of microorganism is determined to be increased when the level (e.g., activity, expression, concentration, level, etc.) of microorganism in the biological sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In certain embodiments, the comparator may be at the level of the relevant biomarkers (e.g., the activity of microorganism, expression of microorganism, concentration of microorganism, level of microorganism, etc.) in a subject not having a disease or disorder associated with increased level (e.g., activity, expression, concentration, level, etc.) of microorganism, a subject not at risk of developing a disease or disorder associated with increased level (e.g., activity, expression, concentration, level, etc.) of microorganism, a population not having a disease or disorder associated with increased level (e.g., activity, expression, concentration, level, etc.) of microorganism, or a population not having a risk of developing a disease or disorder associated with increased level (e.g., activity, expression, concentration, level, etc.) of microorganism.

In some embodiments, the disease or disorder is associated with decreased level (e.g., activity, expression, level, etc.) of at least one microorganism and/or a microbial metabolite.

In one embodiment, the disease or disorder is associated with a decreased or reduced level of microbial metabolite when the level (e.g., activity, expression, concentration, level, etc.) of microbial metabolite is decreased in the biological sample as compared to a comparator. In various embodiments of the methods of the invention, the level (e.g., activity, expression, concentration, level, etc.) of microbial metabolite is determined to be decreased when the relevant biomarkers (e.g., the activity of microbial metabolite, expression of microbial metabolite, concentration of microbial metabolite, level of microbial metabolite, etc.) are differentially expressed as compared to a comparator. In certain embodiments, the comparator may be at the level of the relevant biomarkers (e.g., the activity of microbial metabolite, expression of microbial metabolite, concentration of microbial metabolite, level of microbial metabolite, etc.) in a subject not having a disease or disorder associated with decreased level (e.g., activity, expression, concentration, level, etc.) of microbial metabolite, a subject not at risk of developing a disease or disorder associated with decreased level (e.g., activity, expression, concentration, level, etc.) of microbial metabolite, a population not having a disease or disorder associated with decreased level (e.g., activity, expression, concentration, level, etc.) of microbial metabolite, or a population not having a risk of developing a disease or disorder associated with decreased level (e.g., activity, expression, concentration, level, etc.) of microbial metabolite.

In one embodiment, the disease or disorder is associated with a decreased or reduced level of microorganism when the level (e.g., activity, expression, concentration, level, etc.) of microorganism is decreased in the biological sample as compared to a comparator. In various embodiments of the methods of the invention, the level (e.g., activity, expression, concentration, level, etc.) of microorganism is determined to be decreased when the relevant biomarkers (e.g., the activity of microorganism, expression of microorganism, concentration of microorganism, level of microorganism, etc.) are differentially expressed as compared to a comparator.

In various embodiments of the methods of the invention, the level (e.g., activity, expression, concentration, level, etc.) of microorganism is determined to be decreased when the level of microorganism (e.g., activity, expression, concentration, level, etc.) in the biological sample is decreased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., activity, expression, concentration, level, etc.) of microorganism is determined to be decreased when the level (e.g., activity, expression, concentration, level, etc.) of microorganism in the biological sample is determined to be decreased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In certain embodiments, the comparator may be at the level of the relevant biomarkers (e.g., the activity of microorganism, expression of microorganism, concentration of microorganism, level of microorganism, etc.) in a subject not having a disease or disorder associated with decreased level (e.g., activity, expression, concentration, level, etc.) of microorganism, a subject not at risk of developing a disease or disorder associated with decreased level (e.g., activity, expression, concentration, level, etc.) of microorganism, a population not having a disease or disorder associated with decreased level (e.g., activity, expression, concentration, level, etc.) of microorganism, or a population not having a risk of developing a disease or disorder associated with decreased level (e.g., activity, expression, concentration, level, etc.) of microorganism.

In various embodiments, the disease or disorder is associated with the level (e.g., activity, expression, level, etc.) of at least one microorganism and/or a microbial metabolite is a disease or disorder associated with the level of Escherichia coli LF82, disease or disorder associated with the level of Enterococcus faecalis, disease or disorder associated with the level of Lactobacillis plantarum, disease or disorder associated with the level of Faecalibacterium prauznitzii, disease or disorder associated with the level of Bifidobacterium longum, disease or disorder associated with the level of Bacteroides vulgatus, disease or disorder associated with the level of Ruminococcus gnavus, disease or disorder associated with the level of tyramine, disease or disorder associated with the level of tryptamine, disease or disorder associated with the level of cadaverine, disease or disorder associated with the level of 9,10-methylenehexadecanoic acid, disease or disorder associated with the level of 12-methyltetradecanoic acid (also referred to as 12-methylmyristic acid), disease or disorder associated with the level of 13-methyltetradecanoic acid (also referred to as 13-methylmyristic acid), disease or disorder associated with the level of 14-methylpalmitic acid, disease or disorder associated with the level of 4-hydroxycinnamic acid, disease or disorder associated with the level of anteiso-fatty acid, disease or disorder associated with the level of iso-fatty acid, or any combination thereof.

In various embodiments, the method comprises modulating the level (e.g., activity, expression, level, etc.) of the at least one microorganism and/or a microbial metabolite thereof in the subject. In some embodiments, the method comprises modulating the level of the at least one microorganism in the subject. In some embodiments, the method comprises modulating the level of the at least one microbial metabolite of the microorganism of the present invention in the subject. In some embodiments, the method comprises modulating the level of the at least one microorganism and a microbial metabolite thereof in the subject.

In one embodiment, the method comprises reducing the level (e.g., activity, expression, level, etc.) of the at least one microorganism and/or a microbial metabolite thereof in the subject. In one embodiment, the method comprises reducing the level of the at least one microorganism in the subject. In one embodiment, the method comprises reducing the level of the at least one microbial metabolite of the microorganism in the subject. In one embodiment, the method comprises reducing the level of the at least one microorganism and a microbial metabolite thereof in the subject. For example, in one embodiment, the level (e.g., activity, expression, level, etc.) of microorganism is determined to be a decreased or reduced level of microorganism when the level (e.g., activity, expression, concentration, level, etc.) of microorganism is decreased by at least 1 fold, at least 1.1 fold, at least 1.2 fold, at least 1.3 fold, at least 1.4 fold, or at least 1.5 fold. In one embodiment, the level of microbial metabolite is determined to be a decreased or reduced level of microbial metabolite when the level (e.g., activity, expression, concentration, level, etc.) of microbial metabolite is decreased by at least 1 fold, at least 1.1 fold, at least 1.2 fold, at least 1.3 fold, at least 1.4 fold, or at least 1.5 fold.

In one embodiment, the method comprises administering a therapeutically effective amount of an inhibitor of the at least one microorganism and/or a microbial metabolite thereof to the subject. In one embodiment, the method comprises reducing the level (e.g., activity, expression, level, etc.) of the at least one microorganism and/or a microbial metabolite thereof in the subject by administering a therapeutically effective amount of an inhibitor of the at least one microorganism and/or a microbial metabolite thereof to the subject. In some embodiments, the inhibitor of the at least one microorganism and/or a microbial metabolite thereof is an inhibitor of microorganism, inhibitor of microbial metabolite, inhibitor of microbial metabolite biosynthesis, inhibitor of microbial metabolite synthesis, or any combination thereof. Examples of inhibitors of the at least one microorganism and/or a microbial metabolite thereof include, but are not limited to, a nucleic acid or a fragment thereof, peptide or a fragment thereof, protein or a fragment thereof, fusion protein or a fragment thereof, chemical compound, small molecule, ribozyme, antibody or a fragment thereof, acid, base, therapeutic agent, or any combination thereof.

For example, in some embodiments, the inhibitor of at least one microorganism is an inhibitor of Escherichia coli LF82, an inhibitor of Enterococcus faecalis, an inhibitor of Lactobacillis plantarum, an inhibitor of Faecalibacterium prauznitzii, an inhibitor of Bifidobacterium longum, an inhibitor of Bacteroides vulgatus, an inhibitor of Ruminococcus gnavus, or any combination thereof. In some embodiments, the inhibitor of the at least one microbial metabolite is an inhibitor of tyramine, an inhibitor of tyramine biosynthesis, an inhibitor of tyramine synthesis, an inhibitor of tryptamine, an inhibitor of tryptamine biosynthesis, an inhibitor of tryptamine synthesis, an inhibitor of cadaverine, an inhibitor of cadaverine biosynthesis, an inhibitor of cadaverine synthesis, an inhibitor of 9,10-methylenehexadecanoic acid, an inhibitor of 9,10-methylenehexadecanoic acid biosynthesis, an inhibitor of 9,10-methylenehexadecanoic acid synthesis, an inhibitor of 12-methyltetradecanoic acid (also referred to as 12-methylmyristic acid), an inhibitor of 12-methyltetradecanoic acid (also referred to as 12-methylmyristic acid) biosynthesis, an inhibitor of 12-methyltetradecanoic acid (also referred to as 12-methylmyristic acid) synthesis, an inhibitor of 13-methyltetradecanoic acid (also referred to as 13-methylmyristic acid), an inhibitor of 13-methyltetradecanoic acid (also referred to as 13-methylmyristic acid) biosynthesis, an inhibitor of 13-methyltetradecanoic acid (also referred to as 13-methylmyristic acid) synthesis, an inhibitor of 14-methylpalmitic acid, an inhibitor of 14-methylpalmitic acid biosynthesis, an inhibitor of 14-methylpalmitic acid synthesis, an inhibitor of 4-hydroxycinnamic acid, an inhibitor of 4-hydroxycinnamic acid biosynthesis, an inhibitor of 4-hydroxycinnamic acid synthesis, an inhibitor of anteiso-fatty acid, an inhibitor of anteiso-fatty acid biosynthesis, an inhibitor of anteiso-fatty acid synthesis, an inhibitor of iso-fatty acid, an inhibitor of iso-fatty acid biosynthesis, an inhibitor of iso-fatty acid synthesis, or any combination thereof.

In some embodiments, the method comprises reducing the level (e.g., activity, expression, level, etc.) of the at least one microorganism and/or a microbial metabolite thereof in the subject by modulating the pH. In some embodiments, the method comprises reducing the level (e.g., activity, expression, level, etc.) of the at least one microorganism and/or a microbial metabolite thereof in the subject by increasing the pH. In some embodiments, the method comprises reducing the level (e.g., activity, expression, level, etc.) of the at least one microorganism and/or a microbial metabolite thereof in the subject by decreasing the pH. For example, in some embodiments, the method comprises reducing the level (e.g., activity, expression, level, etc.) of cadaverine in the subject by increasing the pH in the gut of the subject.

In one embodiment, the method comprises increasing the level (e.g., activity, expression, level, etc.) of the at least one microorganism and/or a microbial metabolite thereof in the subject. In one embodiment, the method comprises increasing the level of the at least one microorganism in the subject. In one embodiment, the method comprises increasing the level of the at least one microbial metabolite of the microorganism in the subject. In one embodiment, the method comprises increasing the level of the at least one microorganism and a microbial metabolite thereof in the subject. For example, in one embodiment, the level (e.g., activity, expression, level, etc.) of microorganism is determined to be an increased level of microorganism when the level (e.g., activity, expression, concentration, level, etc.) of microorganism is increased by at least 1 fold, at least 1.1 fold, at least 1.2 fold, at least 1.3 fold, at least 1.4 fold, or at least 1.5 fold. In one embodiment, the level of microbial metabolite is determined to be an increased level of microbial metabolite when the level (e.g., activity, expression, concentration, level, etc.) of microbial metabolite is increased by at least 1 fold, at least 1.1 fold, at least 1.2 fold, at least 1.3 fold, at least 1.4 fold, or at least 1.5 fold.

For example, in various embodiments, the method comprises increasing the level (e.g., activity, expression, level, etc.) of the at least one microorganism and/or a microbial metabolite thereof in the subject by administering at least one microorganism and/or a microbial metabolite thereof to the subject. In some embodiments, the method comprises increasing the level (e.g., activity, expression, level, etc.) of the at least one microorganism and/or a microbial metabolite thereof in the subject by administering a nucleic acid or a fragment thereof, peptide or a fragment thereof, protein or a fragment thereof, fusion protein or a fragment thereof, chemical compound, small molecule, ribozyme, antibody or a fragment thereof, acid, base, therapeutic agent, or any combination thereof, that increase the level (e.g., activity, expression, level, etc.) of the microorganism and/or a microbial metabolite thereof to the subject.

In some embodiments, the method comprises increasing the level (e.g., activity, expression, level, etc.) of the at least one microorganism and/or a microbial metabolite thereof in the subject by modulating the pH. In some embodiments, the method comprises increasing the level (e.g., activity, expression, level, etc.) of the at least one microorganism and/or a microbial metabolite thereof in the subject by increasing the pH. In some embodiments, the method comprises increasing the level (e.g., activity, expression, level, etc.) of the at least one microorganism and/or a microbial metabolite thereof in the subject by decreasing the pH. For example, in some embodiments, the method comprises icreasing the level (e.g., activity, expression, level, etc.) of cadaverine in the subject by decreasing the pH in the gut of the subject.

In various embodiments, the disease or disorder associated with the at least one microorganism and/or a microbial metabolite thereof is an inflammatory disease or disorder, autoimmune disease or disorder, inflammatory bowel disease, Crohn's disease, Ulcerative Colitis, or any combination thereof. For example, in one embodiment, the present invention provides a method of treating or preventing an inflammatory disease or disorder in a subject in need thereof by administering an inhibitor of cadaverine, inhibitor of cadaverine synthesis, inhibitor of cadaverine biosynthesis, or any combination thereof to the subject. In another embodiment, the present invention provides a method of treating or preventing Crohn's disease in a subject in need thereof by administering an inhibitor of cadaverine, inhibitor of cadaverine synthesis, inhibitor of cadaverine biosynthesis, or any combination thereof to the subject. In another embodiment, the present invention provides a method of treating or preventing an autoimmune disease or disorder in a subject in need thereof by administering an inhibitor of 9,10-methylenehexadecanoic acid, inhibitor of 9,10-methylenehexadecanoic acid biosynthesis, inhibitor of 9,10-methylenehexadecanoic acid synthesis, or any combination thereof to the subj ect.

In some embodiments, the method of treatment comprises monitoring the biomarker levels during the course of treatment of a disease or disorder. In some embodiments, the method of treatment comprises an assessment of the effectiveness of the treatment regimen for a disease or disorder, such as cancer, by detecting one or more biomarkers in an effective amount from samples obtained from a subject over time and comparing the amount of biomarker or biomarkers detected. In some embodiments, a first sample is obtained prior to the subject receiving treatment and one or more subsequent samples are taken after or during treatment of the subject. In some embodiments, changes in biomarker levels over time provide an indication of effectiveness of the therapy.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

In various exemplary embodiments, effecting a therapy comprises administering a disease-modulating drug to the subject. The subject may be treated with one or more drugs until altered levels of the measured biomarkers return closer to the baseline value measured in a population not having a disease or disorder, or showing improvements in disease biomarkers as a result of treatment with a drug. Additionally, improvements related to a changed level of a biomarker or clinical parameter may be the result of treatment with a disease-modulating drug.

Any drug or any combination of drugs disclosed herein may be administered to a subject to treat a disease. The drugs herein can be formulated in any number of ways, often according to various known formulations in the art or as disclosed or referenced herein.

In various embodiments, any drug or any combination of drugs disclosed herein is not administered to a subject to treat a disease. In these embodiments, the practitioner may refrain from administering the drug or any combination of drugs, may recommend that the subject not be administered the drug or any combination of drugs or may prevent the subject from being administered the drug or any combination of drugs.

In various embodiments, one or more additional drugs may be optionally administered in addition to those that are recommended or have been administered.

### Therapeutics

The invention also includes therapeutic activators and therapeutic inhibitors of the microbial metabolites and host receptors identified by the methods of the invention described herein. One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

### Kits

The present invention also pertains to kits useful in the methods of the invention. Such kits comprise various combinations of components useful in any of the methods described elsewhere herein, including for example, materials for screening microbial metabolites, materials for screening host receptors, materials for preventing or treating a disease or disorder associated with the level (e.g., activity, expression, level, etc.) of at least one receptor listed in FIG. 2B, FIG. 15, and Table 1, materials for preventing or treating a disease or disorder associated with the level (e.g., activity, expression, level, etc.) of at least one microorganism and/or a microbial metabolite, and instructional material. For example, in one embodiment, the kit comprises components useful for the screening microbial metabolites. In another embodiment, the kit comprises components useful for screening host receptors. In a further embodiment, the kit comprises components useful for preventing or treating a disease or disorder associated with the level (e.g., activity, expression, level, etc.) of at least one receptor listed in FIG. 2B, FIG. 15, and Table 1. In a further embodiment, the kit comprises components useful for preventing or treating a disease or disorder associated with the level (e.g., activity, expression, level, etc.) of at least one microorganism and/or a microbial metabolite.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Characterization of Microbiota Encoding GPCR Agonist

This invention describes, in part, the identification of the microbiota host interaction by systematically screening metabolites produced by individual members of a simplified human microbiota for the ability to agonize host receptors. Large-scale functional screening of small molecules produced by individual members of a simplified human microbiota was used to identify microbial encoded metabolites that stimulate GPCR. The resulting interaction map provides evidence, at the molecular level, for the existence of a complex network of microbial-host interactions, many of which involve receptors associated with immunological, neurological or cardiovascular disorders. The detailed characterization of interactions predicted by this analysis led to the discovery of multiple previously unrecognized microbiota encoded GPCR agonists. The structures of the active molecules identified in this invention suggest that simple bacterial metabolites arising from primary metabolic processes are likely to broadly impact human physiology.

The majority of U.S. Food and Drug Administration (FDA)-approved drugs target just three receptor classes: GPCRs, ion channels, and nuclear hormone receptors (Santos R et al., 2017, Nat Rev Drug Discov 16:19-34). Many endogenous eukaryotic signaling metabolites target these same receptor families (Rosenbaum et al., 2009, Nature 459:356-363). Their dominant roles translating small molecules into biological responses suggested that these receptors will be important to the modulation of human physiology by metabolites encoded within the microbiota. To directly couple microbiota encoded small molecules to specific biological activities, the responses of 241 GPCRs were mapped to a library of metabolites generated by members of a simplified human microbiota (SIHUMI) consortium. This invention focuses in part on a consortium composed of seven taxonomically diverse bacteria representing commensal, beneficial, and pathogenic species with a well-established history of stably colonizing human and murine gastrointestinal tracts (FIG. 2A) (Eun C S et al., 2014, Infect Immun, 82:2239-2246).

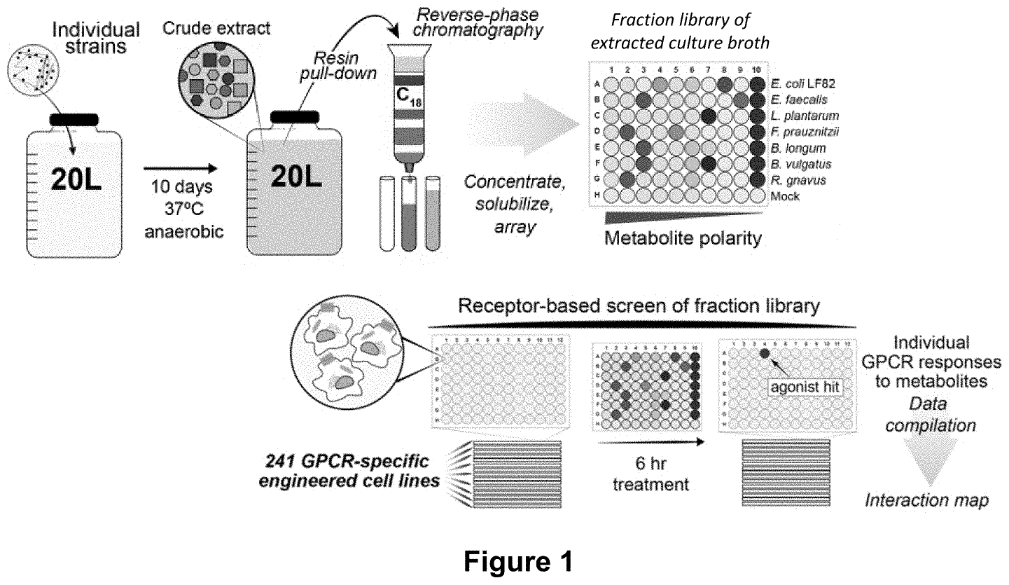

Individual bacteria from the SIHUMI consortium were fermented under anaerobic conditions in 20 L culture vessels, along with a media control containing no bacteria (FIG. 1). After 10 days, the extracellular metabolites were extracted and the crude mixture was partitioned into metabolite-rich fractions using reversed phased flash chromatography. The pre-fractionation process simplifies each fraction, thereby improving the signal in the primary screen and facilitating the downstream isolation of bioactive compounds. The fraction library was screened for agonist activity against individual GPCRs using a luminescence-based β-arrestin recruitment assay. Receptors designated with orphan status as well as extensively studied receptor families both responded substantially to bacterial derived fractions over media control fractions (FIG. 2A). Notably, a large number of receptors that were most strongly agonized by microbiota encoded metabolites are also targeted by FDA approved drugs and expressed in microbe-rich body sites (FIG. 2B).

Bioassay-guided isolation of pure metabolites from the large-scale bacterial culture broth extracts followed by de novo structure elucidation was used to identify GPCR-active metabolites. In parallel to these in vitro analyses, high-resolution mass spectrometry-based metabolomics was used to compare metabolites present in cecum from germ-free mice colonized with the SIHUMI consortium to cecum obtained from non-colonized mice. Coupling these studies enabled the identification of functional metabolites that accumulate in mammalian cecum in a microbiota-dependent manner. The following vignettes highlight the characterization of metabolites associated with a subset of the interactions revealed in the functional mapping of the SIHUMI consortium.

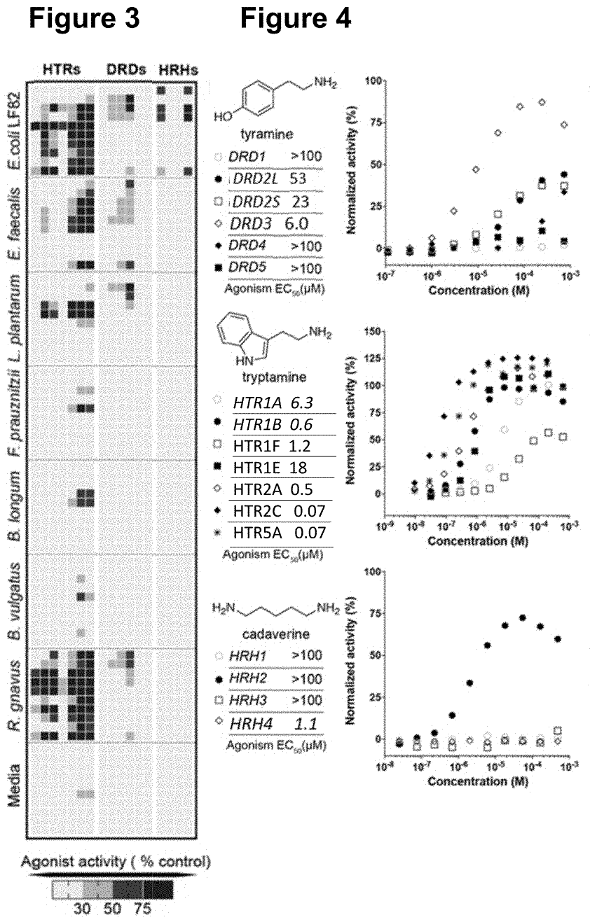

The GPCR interaction map revealed numerous bacterial fractions that significantly agonized neurotransmitter receptors, a key component of the gut brain axis (FIG. 2B) (Mittal Ret al., 2017, Journal of Cellular Physiology 232:2359-2372). In fractions from multiple bacterial species, agonism of a subset of D2-type dopamine receptors (DRDs), DRD2 and DRD3 were observed (FIG. 3). Bioassay-guided isolation led to the biogenic amine tyramine, whose agonism of DRD2 and DRD3 was validated using a synthetic standard (FIG. 4). While no biological significance had been assigned to the microbiota dependent production of tyramine in animal models, the results suggest that it accumulates in the gastrointestinal tract to a sufficient level to agonize D2 subtype DRDs (Sridharan GV et al., 2014, Nature Communications 5:5492). Similarly, agonism of serotonin receptors (HTRs) was also observed for fractions from various species (FIG. 3). Isolation of the active metabolite yielded tryptamine, which, similar to tyramine, was produced in varying quantities by multiple members of the consortium in vitro (FIG. 4). The observation that human associated bacteria produce tryptamine that can modulate serotonin receptors, which arose directly from the receptor activity map (FIG. 2A), corroborates similar observations from hypothesis-driven studies (Bhattarai Y et al., 2018, Cell Host & Microbe 23:775-785).

In contrast to the broad activation seen for DRDs and HTRs across the majority of bacteria in the consortium, a specific response to fractions from the pathogen Escherichia coli LF82 was detected for a member of the histamine receptor (HRH) family, HRH4. (FIG. 3). The polyamine cadaverine was identified as the major metabolite responsible for HRH4 activation (FIG. 4). In addition to cadaverine a number of structurally related synthetic polyamines were tested for HRH4 activity. Agmatine and putrescine selectively agonized HRH4 but to a lesser extent than cadaverine, and spermidine showed no HRH activity across the entire receptor family. Some bacteria, including E. coli LF82, are known to increase the production of cadaverine in acidic environments, as they harbor gene clusters that are induced in response to extracellular pHs below 6.8 (Ma Wet al., 2017, Engineering 3:308-317). Interestingly, although histamine receptor subtypes differ in their associated functions and distribution throughout the body, HRH4 in the gastrointestinal tract has been linked to inflammatory responses related to inflammatory bowel diseases and cancer (Coruzzi GM et al., 2012, Front Biosci (Schol Ed) 4:226-239).

A growing number of studies have uncovered connections between gut microbiota and the nervous system (Dinan TG et al., 2017, Clin North Am 46:77-89; Sharon G et al., 2016, Cell 167:915-932). The exploration of microbiota encoded neurotransmitter receptor agonists expands the mechanistic evidence for simple biogenic amines serving as potentially widespread modulators of the gut-brain axis (Pugin B et al., 2017, Microbial Ecology in Health and Disease 28:1353881). These data imply that microbiota-dependent dopaminergic, serotonergic and histaminergic responses likely represent general signaling events in the gastrointestinal tract with varying activation profiles depending on the specific collection of bacteria present in an individual's microbiome.

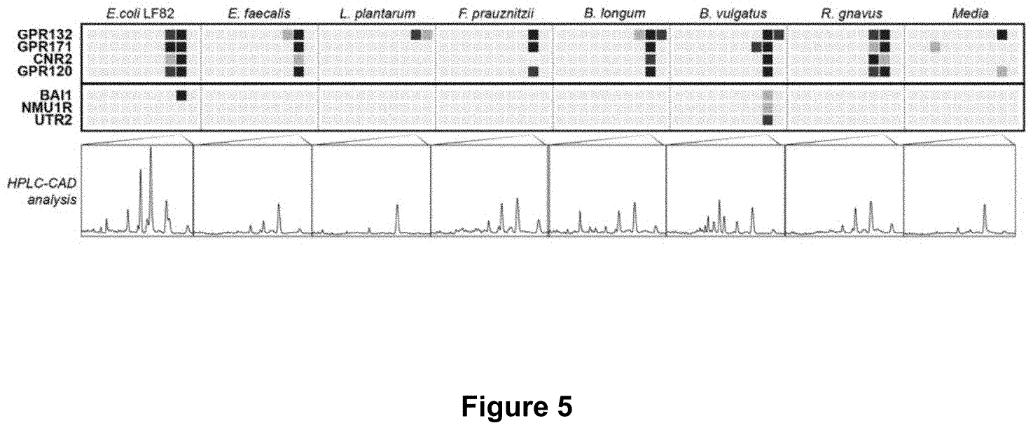

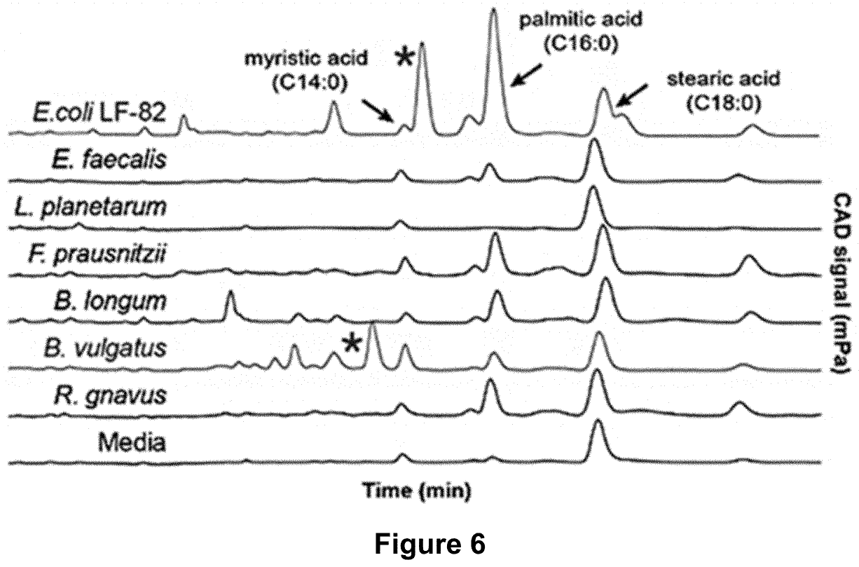

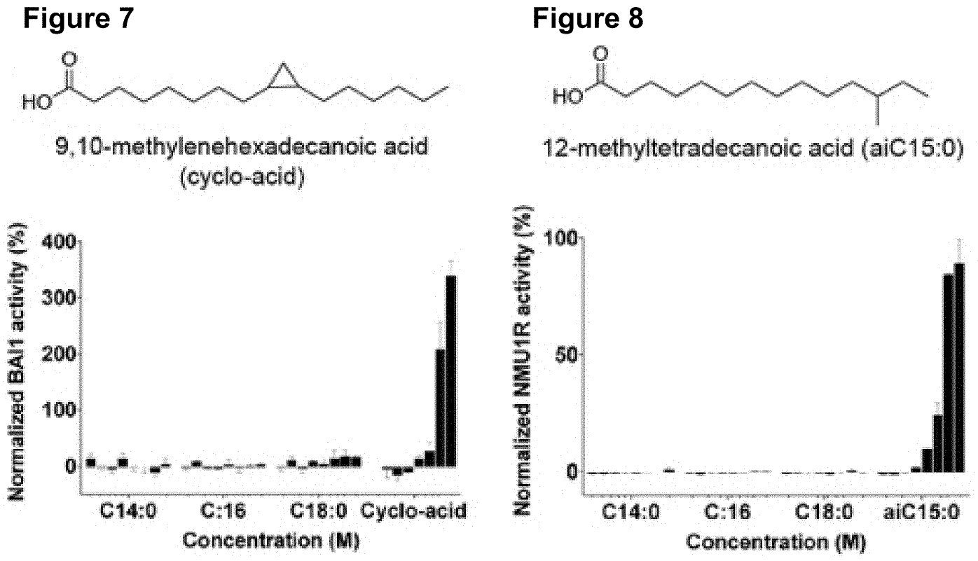

Lipids, which represent diverse GPCR active ligands (Round J L et al., 2011, Science 332:974-977; An D et al., 2014, Cell 156:123-133), predominantly elute very late in the fractionation protocol. Based on the receptor interaction map, GPCRs were initially classified as lipid responsive if they were agonized by the late lipid-enriched fractions of the extract library (FIG. 5). A subset of receptors, including GPR120, CNR2, GPR171, GPR132, responded broadly to the lipid fraction from most of the consortium, whereas other responses were specific to particular species (BAI1, NMU1R, UTR2). HPLC-charged aerosol detection analysis of the lipid fractions indicated they contained not only mixtures of simple saturated fatty acids but also other more complex lipid species (FIG. 6). Marrying unique receptor activity profiles with unique lipid signals guided us to previously unrecognized bacteria encoded GPCR agonists. The GPCR brain angiogenesis factor 1 (BAI1) was agonized by lipid fractions from Gram-negative bacteria in the consortium (FIG. 5). The BAI1 agonist was identified as the cyclopropyl-containing lipid 9,10-methylenehexadecanoic acid (3 μg/mL), a fatty acid derivative that is enriched in Gram-negative as well as mycolic acid bacteria (FIG. 7) (Wessjohann L A et al., 2003, Chem Rev 103:1625-1648). Synthetic 9,10-methylenehexadecanoic acid, but no saturated lipids that were tested, agonized BAIL confirming the specificity of the receptor reflected in the activity map (FIG. 7). Macrophages use BAD as a pattern recognition receptor to sense Gram-negative bacteria and induce selective phagocytosis and antimicrobial responses; 9,10-methylenehexadecanoic acid may represent a previously unrecognized recognition motif for innate immune responses (Das S et al., 2011, Proceedings of the National Academy of Sciences 108:2136-2141; Ito J et al., 2009, Cell and Tissue Research 338:257).

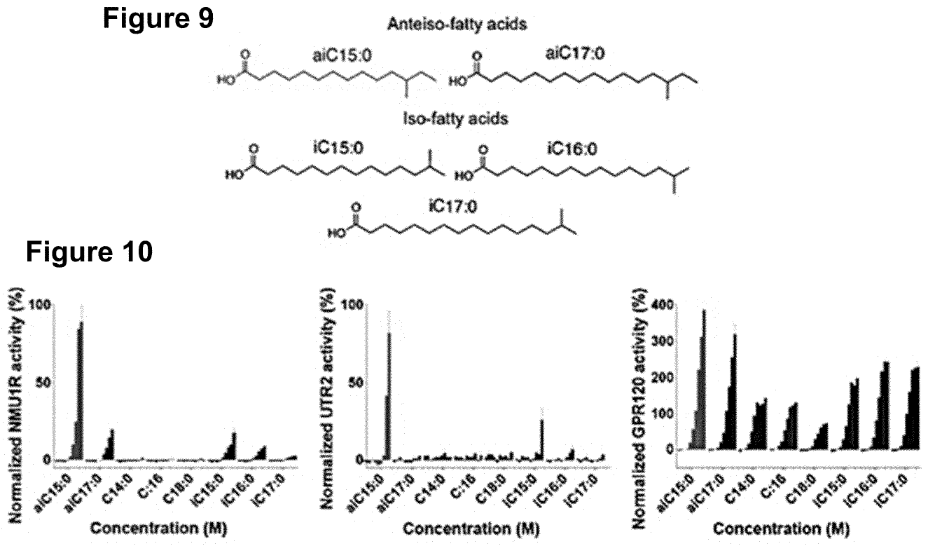

Two peptide receptors NMU1R (neuromedin receptor 1), which mediates satiety and peristalsis in the gut (Howard A D et al., 2000, Nature 406:70-74) and the vasoregulating urotensin 2 receptor (UTR2) responded specifically to lipid fractions generated from Bacteroides vulgatus (FIG. 5). Isolation of the active metabolite yielded the ante-iso methyl branched fatty acid, 12-methylmyristate (aiC15:0) (FIG. 8 and FIG. 9). Both synthetic and natural 12-methylmyristate, but no simple fatty acids that were tested, agonized NMU1R (EC50 32 μg/mL) and UTR2 (EC50 49 μg/mL). Lipid sensitivity of NMU1R and UTR2 appears specific to 12-methylmyristate, as fatty acids with even slightly modified branching patterns (iC15:0) or carbon chain length (aiC17:0) displayed minimal agonist activity (FIG. 10). Roughly 10% of bacteria have lipid pools enriched in branched chain fatty acids (Kaneda T, 1991, Microbiological Reviews 55:288-302). B. vulgatus, is among those bacteria enriched in branched chain fatty acids and maintains 12-methylmyristate as ˜30% of its total fatty acid repertoire (Kaneda T, 1991, Microbiological Reviews 55:288-302). Bacteria are known to produce diverse and often times taxa specific, collections of lipids (Kaneda T, 1991, Microbiological Reviews 55:288-302; Mayberry W R et al., 1982, International Journal of Systematic and Evolutionary Microbiology, 32: 21-27). The examples described here represent a subset of lipid activation data generated from a small consortium, suggesting the potential for markedly different receptor activation profiles and hence biological consequences depending on the specific lipid signature encoded by an individual's microbiome.

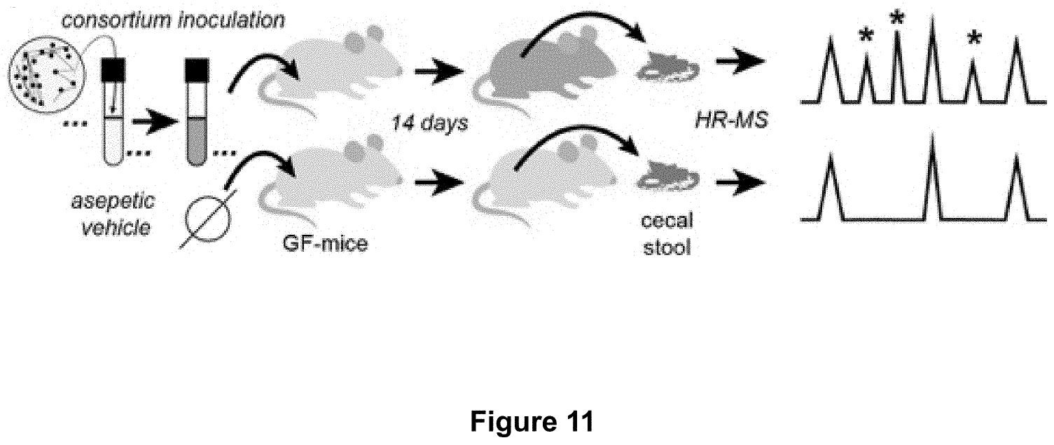

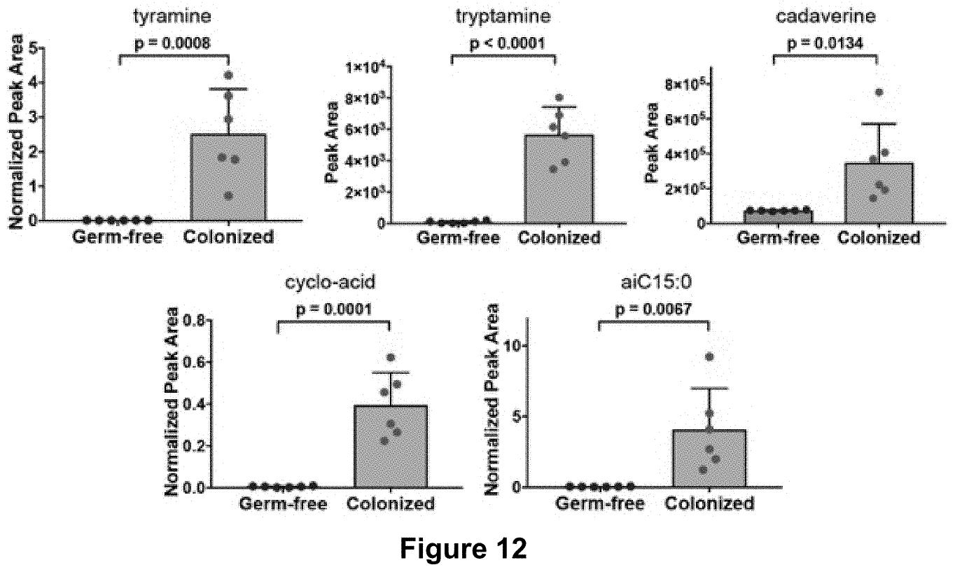

The analysis of cecum from germ-free mice colonized with the SIHUMI consortium revealed that all metabolites described here were enriched in these mice compared to their abiotic counterparts (FIG. 11 and FIG. 12), suggesting parallel biosynthesis in laboratory grown mono-cultures and the consortium in vivo. The metabolites described here add to a growing list of structurally simple molecules that are capable of modulating human signaling pathways that underlie diverse clinically relevant areas of mammalian physiology including immune recognition, neurotransmission, and inflammation (Brown J M et al., 2017, J Biol Chem 292:8560-8568; Pugin B et al., 2017, Microbial Ecology in Health and Disease 28:1353881; Rooks M G et al., 2016, Nat Rev Immunol, 16:341-352). The biosynthetic simplicity of these metabolites combined with their abundant starting materials likely drives their high titers in the gut and potential broad biological relevance. Advancements in laboratory culturing techniques now allow for a majority of gut bacteria to be cultured from fecal samples (Forster SC et al., 2019, Nature Biotechnology 37:186-192). Systematically expanding functional screening to include additional cultured bacteria, growth conditions, and receptor families will undoubtedly provide even deeper insight into the influence the microbiome has on its human host. Above describe overlap between therapeutically targeted receptors and those agonized by metabolites from a minimized microbiome (FIG. 2B) suggests that leveraging these interactions is likely to have diverse therapeutically relevant outcomes.

As such, this analysis revealed a complex network of metabolite host receptor interactions and guided the characterization of multiple microbiota encoded agonists of GPCR associated with the nervous and immune systems, among others. Collectively, these uncovered metabolite-receptor pairs indicate that diverse aspects of human health are potentially modulated by structurally diverse simple metabolites arising from primary bacterial metabolism.

The materials and methods are now described.

### Methods

Media Construction: LBM Media

LBM media was derivative of a media recipe previously utilized in the laboratory. For 1 L media: 17 g/L brain heart infusion, 5 g/L yeast extract, 200 mg MgSO4.7H2O, 100 mg MnCl2.4H2O up was added to 800 mL deionized water and autoclaved for 30 minutes liquid cycle. After coming to room temp, supplements were added (final concentrations: 5 μg/L hemin, 1 g/L maltose, 1 g/L cellobiose, and 0.5 g/L L-cysteine), which can be made ahead of time and stored, protected from light, at −20° C. in aliquots, except hemin which can be stored at 4° C. and L-cysteine which should be made fresh. Autoclaved deionized water was used to bring final volume to 1 L. For culturing anaerobes: media was placed in anaerobic chambers for at least 48 hrs to allow diffusion with anaerobic gas.
Cultivation of Bacteria

Bacterial strains of the SIHUMI consortium were used. Cultures of <1 L: Anaerobic bacteria were cultured in an incubator set to 37° C. placed inside of vinyl anaerobic chamber (Coy) with a gas mix of 5% CO2, 5% H2, and 90% N2. Cultures of >1 L: When cultivating bacteria for construction of bacterial extract library, bacteria were inoculated in 1 L or 2 L media bottles (Chemglass) inside anaerobic chamber, then sealed with anaerobic septa (Chemglass) and moved into large walk-in 37° C. incubator constructed from a 6×6×12 light protective tent (APOLLO® HORTICULTURE) outfitted with a regulator (INKBIRD®), heat source (VORNADO®), and ventilation system (IPOWER®). Freezer stocks of the SIHUMI cohort were generously donated by the Mucida laboratory (Rockefeller University). Freezer stocks were thawed and bacteria were cultivated overnight in LBM media until turbid. These bacteria were streaked onto LBM agar plates and upon growth, single colonies were picked, cultivated overnight, and genotyped (GeneWiz). Upon confirmation of genetic identity, these same cultures were used to generate colony 20% glycerol stocks that would be used for the entirety of the study. Bacteria specific primers were designed to allow for PCR-based identification of each specific strain.

Escherichia coli LF-82
(F: GTTAATACCTTTGCTCATTGA (SEQ ID NO: 1),
R: ACCAGGGTATATAATCCTGTT (SEQ ID NO: 2), 340 bp);
Faecahbacterium prausnitzii DSM 17677
(F: CCCTTCAGTGCCGCAGT (SEQ ID NO: 3),
R: GTCGCAGGATGTCAAGAC (SEQ ID NO: 4), 158 bp);
Enterococcus faecalis OG1RF
(F: CCCTTATTGTTAGTTGCCATCATT (SEQ ID NO: 5),
R: ACTCGTTGTACTTCCCATTGT (SEQ ID NO: 6), 144 bp);
Bifidobacterium longum ATCC 15707
(F: GGGTGGTAATGCCGGATG (SEQ ID NO: 7),
R: TAAGCGATGGACTTTCACACC (SEQ ID NO: 8), 442 bp);
Lactobacillus plantarum ATCC BAA-793
(F: AGCAGTAGGGAATCTTCCA (SEQ ID NO: 9),
R: CACCGCTACACATGGAG (SEQ ID NO: 10), 341 bp);
Bacteroides vulgatus ATCC 8482
(F: GGTGTCGGCTTAAGTGCCAT (SEQ ID NO: 11),
R: CGGAYGTAAGGGCCGTGC (SEQ ID NO: 12), 140 bp);
Ruminococcus gnavus ATCC 29149
(F: CGGTACCTGACTAAGAAGC (SEQ ID NO: 13),
R: AGTTTYATTCTTGCGAACG (SEQ ID NO: 14), 429 bp)

For large scale fermentations the following protocol was used: Bacterial stocks were thawed and used to inoculate 5 mL LBM liquid cultures that were cultivated overnight. The next day, species specific primers were used to confirm identity (as described below) and upon passing purity check, these 5 mL cultures were used to inoculate 500 mL LBM ata ˜1:100 ratio. After turbidity was reached, an aliquot of the 500 mL culture was removed and PCR was performed with universal 16s rRNA primers 27F (AGAGTTTGATCMTGGCTCAG) and 1492R (GGTTACCTTGTTACGACTT). The CR product was subject to Sanger sequencing (GeneWiz) and upon passing inspection for the correct species, the 500 mL culture was used to inoculate 12 L of LBM media at a 1:100 inoculation ratio. The 20 L cultures were cultivated, protected from light at 37° C., for 10 days without shaking. Amerlite XAD-7HP (Sigma Aldrich) was aliquoted in 20 g increments and activated by soaking in methanol for 10 minutes, followed by 5 washes with deionized water to remove excess methanol. After 10 days, activated Amberlite XAD-7HP was added to the cultures (20 g dry weight/L) and the slurries were gently shaken (90 rpm) on a tabletop shaker for 4 hrs. After incubation with the cultures, the resin was removed via cheese-cloth filtration and the collected resin. alongside the cheese-cloth, was placed inside a 1 L Fernbach flask to which 1.5 L acetone was added. This acetone elution was allowed to occur for 2 hrs with shaking (150 rpm), after which the organic solvent was collected and fresh acetone, of equivalent volume, was added. This second elution was allowed to occur overnight with light shaking at 22° C. Both elutions were added together and solvent was removed via rotary evaporation (Buchi) at 25° C. to afford the dry crude extract, which was stored at -20° C. until fractionated, as detailed below.
Fractionation of Bacterial Extracts

Crude extracts (˜1-3 g/12L) were re-suspended in ˜300 mL methanol and the soluble material was decanted into a 500 mL round bottom flask (rbf). Free C18 resin (2-3 g) was added and the slurry was evaporated under reduced pressure using a rotary evaporator with temperature set to 25° C. (Buchi). The dry material was collected from the rbf, packed semi-tightly into a 50 g cartridge, and capped with a passive frit (Teledyne). This material was chromatographed over a 150 g C18 Gold (Teledyne ISCO) using a solvent system of water (Solvent A) and methanol (Solvent B), with no acid added, with the following conditions. 5 column volumes (CV) of 5% B, 5% B to 99% B over 10 CV, flush with 10 CV 99% B. All flow-through was collected in 50 mL tubes and combined as follows:

Solvent was evaporated using an SPD-2010 speedvac (Thermo Scientific) with a RH-19 rotor (Thermo Scientific) and the resulting dry material was weighed and resuspended at 100 mg/mL using ACS grade DMSO (Fisher Scientific). Of this solution, 250 μL was removed and added to 250 μL DMSO to create 500 μL 50 mg/mL solution; this solution was aliquoted into various sizes of 96-well plates for facile thawing and biological testing at a later time. The remaining 100 mg/mL solution was stored at -80° C. until validation studies required material for bio-assay guided fractionation.
GPCR Assays

Compound plates were shipped on dry ice to Eurofins DiscoverX and the gperMAX and orphanMAX panels were run on all 80 samples in singleton. More information about the PathHunter assays can be found on the Eurofins DiscoverX website.
High-Resolution Mass Spectrometry of Purified Compounds

High Resolution Mass Spectrometry was acquired on a C18 column (Thermo Acclaim 120 C18, 2.1×150 mM) using a Dionex U-3000 HPLC system connected to an LTQ-Orbitrap Mass Spectrometer (Thermo-Fisher).
Murine Work

All experimental procedures were approved by the Animal Care and Use Committee of The Icahn School of Medicine at Mount Sinai (PI Cohen IACUC-2016-0491). Germ free C57BL/6 mice were maintained in sterile isolators with autoclaved food and water in the Gnotobiotic Facility of the Faith Lab at Mount Sinai. 6-week-old mice were used for all experiments (3M and 3F in the treatment group, 5M and 1F in the control group). The treatment group was colonized with the SIHUMI whereas the control group was left germ free. For colonization studies 5 ml of an overnight culture in LBM media of the SIHUMI (treatment group) was centrifuged at 500×g for 2 minutes, the supernatant was decanted and the cells were resuspended in 2 ml of sterile PBS. Germ free mice were gavaged with 100 μL of bacterial culture immediately upon removal from sterile isolators. Colonization was confirmed by collection of fecal pellets after 3 days. Crude DNA was extracted from fecal pellets per protocol (ZymoBIOMICS DNA/RNA Miniprep Kit) and colonization conformed by targeted PCR of each strain using specific primers as detailed above. After colonization mice were housed in specific-pathogen-free conditions and fed with autoclaved water and food. After colonization for 7 days the mice were euthanized and samples were collected for analysis. 200 mg of cecal contents were collected from each mouse and placed immediately at −80° c. 100 mg pieces of cecal tissue were taken, placed in RNAlater (Qiagen) and transferred to −80° c. The animal experiments were not randomized and the investigators were not blinded to the allocation during experiments and outcome assessment. No statistical methods were used to predetermine sample size. All mice which completed the experiments were analyzed.
Metabolite Quantitation by Mass Spectrometry

Cecal samples were weighed into 2 mL microtubes containing 2.8 mm ceramic beads (Omni International) and resuspended to a final concentration of 100 mg/mL using 80:20 methanol:water containing phenol-d7, palmitic acid-d31 and 13C,15N-amino acid internal standards (Cambridge Isotope Laboratories). Homogenization was using a Bead Ruptor (Omni International) at 6 m/s for 30 s for 6 cycles, at 4° C. Samples were centrifuged for 20 minutes at 20,000×g at 4° C. and then divided for 3 analytical methods.

Method 1: GC-nCI-MS with PFB Derivatization

100 μL of cecal extract was added to 100 μL of 100 mM borate Buffer (pH 10), 400 μL of 100 mM pentafluorobenzyl bromide (Thermo Scientific) in acetone (Fisher), and 400 μL of cyclohexane (Acros Organics) in a sealed autosampler vial. Samples were heated to 65° C. for 1 hour with shaking. After cooling to room temperature and allowing the layers to separate, 100 μL of the cyclohexane upper phase was transferred to autosampler vial containing a glass insert and sealed. Analyzed was using an GC-MS (Agilent 7890A GC system, Agilent 5975C MS detector) operating in negative chemical ionization mode, using a DB-5MS column (30 m×0.25 mm, 0.25, 0.25 μm; Agilent Technologies), methane as the reagent gas and 1 μL split injection (1:5 split ratio). Raw peak areas for aromatic analytes (tyramine and hydrocinnamic acid) were normalized to phenol-d7 internal standard and lipid analytes (9,10-methylenehexadecanoic acid and 12-methyltetradecanoic acid) were normalized to palmitic acid-d31 internal standard. Data analysis was performed using MassHunter Quantitative Analysis software (version B.09, Agilent Technologies).

Method 2: LC Triple Quadrupole with Reverse Phase Chromatography

200 μL of extract was dried using a vacuum concentrator (Genevac) and resuspended in 400 μL 50:50 methanol:water, clarified by centrifugation and analyzed suing reverse phase chromatography coupled to TSQ Vantage triple quadupole mass spectrometer with

HESI II source. LC separation was using an HSS T3 column (100×2.1 mm, 1.8 pm particle size, Waters) and Agilent 1260 binary pump. Mobile phase A was 0.1% formic acid in water and Mobile phase B was 0.1% formic acid in acetonitrile. The gradient was 0 min, 0% B; 2 min, 0% B; 5 min, 12% B; 7 min, 70% B; 8.5 min, 97% B, 11.5min, 97% B with 3.5 min of re-equilibration time. LC parameters were: flow rate 300 μL/min, injection volume 15μ and column temperature 35° C. The mass spectrometer was operated in positive ionization with transitions for tyrptamine (m/z 161.1→115.1, CE 30V*; 161.1→144.1, CE 4 V) and nicotinic acid (m/z 124.1→80.1, CE 18 V*; 124.1→78.1, CE 19V), with * indicating the primary transition used for quantitation. MS parameters were: capillary temp: 300° C.; vaporizer temp: 350° C.; sheath gas: 50; aux gas: 30; spray voltage 4000 V. Data was acquired and analyzed using TraceFinder software (version 4.1, Thermo Scientific) confirmed by comparison with authentic standards.

Method 3: LC Q-TOF with HILIC Chromatography

Samples were prepared as for Method 2 but then resuspended in 200 μL 60:40 acetonitrile:water and analyzed by hydrophilic interaction chromatography (HILIC) coupled to the 6545 Q-TOF mass spectrometer with Dual JetStream source (Agilent). The LC separation was using an Acquity UPLC BEH Amide column (150 m×2.1 mm, 1.7 pm particle size, Waters) and Agilent 1290 Infinity II binary pump. Mobile phase A was 90:10 water:acetonitrile with 10 mM ammonium acetate and 0.2% acetic acid, and mobile phase B was 10:90 wate:acetonitrile with 10 mM ammonium acetate and 0.2% acetic acid. The gradient was 0 min, 95% B; 9 min, 70% B; 10 min, 40% B; 13 min, 30% B; 15 min, 95% B. LC parameters were: flow rate 400 μL/min, column temperature 40° C., and injection volume 5 μL. The mass spectrometer was operated in positive ionization mode. MS parameters were: gas temp: 325° C.; gas flow: 10 L/min; nebulizer pressure: 35 psig; sheath gas temp: 400° C.; sheath gas flow: 12 L/min; VCap: 4,000 V; fragmentor: 125 V. Active reference mass correction was done through a second nebulizer using masses with m/z: 12.050873 and 922.009798. Data were acquired over m/z range 50-1700 and analyzed using MassHunter Profinder software (version B.09, Agilent) and confirmed by comparison with a cadaverine authentic standard. Compiling these data sets in GraphPad Prizm was then used to derive p-values. Unpaired t test (two-tailed) were used.
Tyramine

Fraction 3 from E. coli LF82 was chosen as the pilot fraction for the dopamine receptors. 1 mL of Fraction 3 (100 mg/mL in DMSO) was dried down resuspended in 1 mL 50/50 MeOH:H20 and injected in 50 μL increments onto a semi-preparative 250×10 mm Luna® Omega 2.6 uM Polar C18 LC column on an Agilent 1100 HPLC with a solvent system where Solvent A was H2O+0.1% formic acid and Solvent B was CH3CN+0.1% formic acid. The chromatographic method was as follows: 0% B for 5 CV, then up to 90% B over 15 CV, with a 5 CV hold at 90% B. Peak detection and fraction collection was driven by UV absorbance at 210 nm, 254 nm, 280 nm, and 330 nm. Fractions were collected and re-assayed against DRD3 to guide further purification. The active fraction was further purified using a 150×10 mm Kinetix® 5μm Biphenyl 100A LC column. A single resulting fraction retained activity and this compounds was identified as tyramine by NMR and HRMS (LC-HRMS-ESI (m/z): [M+H]+calcd for C8H11NO, 138.0841; found 138.0911). Tyramine: 1H NMR (DMSO-d6, 600 MHz): δH 7.02 (2H, d, J=8.5 Hz), 6.70 (2H, d, J=8.5 Hz), 2.89(2, t, J=7.6 Hz), 2.71 (2H, t, J=7.6 Hz). 13C NMR (DMSO-d6, 151 MHz): δC 156.1 (1C, s), 129.5 (2C, s), 115.3 (2C, s), 40.8 (1C, s), 33.45 (1C, s).
Tryptamine

A single fraction from the fermentation of Ruminococcus gnavus was chosen as a pilot fraction to find serotonin active compounds which could then be assessed in other bacteria. 1 mL of Fraction 5 solution (100 mg/mL in DMSO) was dried down, resuspended in 1 mL 50/50 MeOH:H20, and injected in 50 μL increments onto a semi-preparative 250×10 mm Luna® Omega 2.6 μM Polar C18 LC column on an Agilent 1100 HPLC with a solvent system where Solvent A was H2O+0.1% formic acid and Solvent B was CH3CN+0.1% formic acid. The chromatographic method was as follows: 0% B for 5 CV, then up to 90% B over 15 CV, with a 5 CV hold at 90% B. Peak detection and fraction collection was driven by UV absorbance at 210 nm, 254 nm, 280 nm, and 330 nm. Fractions were collected and re-assayed against HTR5A to guide further purification. The active fraction (41 mg) was ˜90% tryptamine as evident by NMR and HRMS (LC-HRMS-ESI (m/z): [M+H]+ calcd for C8H11NO, 161.1000; found 161.1071). Tryptamine: 1H NMR (DMSO-d6, 600 MHz): δH 7.54 (1H, d, J=7.9 Hz), 7.20 (1H, d, 2.0 Hz), 7.08 (1H, t, 7.6 Hz), 7.00 (1H, t, 7.6 Hz), 3.01 (2H, dd, 8.5 Hz, 7.1 Hz), 2.93 (2H, dd, 8.8, 6.2 Hz). 13C NMR (DMSO-d6, 151 MHz): δC 136.3 (1C, s), 126.9 (1C, s), 123.2 (1C, s), 121.1 (1C, s), 118.4 (1C, s), 118.1 (1C, s), 111.5 (1C, s), 110.1 (1C, s), 40.0 (1C, s), 24.6 (1C, s).
Cadaverine

Fraction 4 from E. coli LF82 was chosen as the pilot fraction for the histamine receptors. 1 mL of Fraction 4 (100 mg/mL in DMSO) was dried down resuspended in 1 mL H2O and injected in 50 μL increments onto a semi-preparative 250×10 mm Luna® Omega 2.6 uM Polar C18 LC column on an Agilent 1100 HPLC with a solvent system where Solvent A was H2O+0.1% formic acid and Solvent B was CH3CN+0.1% formic acid. The chromatographic method was as follows: 0% B for 10 CV, then up to 90% B over 5 CV, with a 3 CV hold at 90% B. Peak detection and fraction collection was driven by charged aerosol detection using a Corona Veo (ThermoFisher Scientific) after UV proved to not be useful. Fractions were collected and re-assayed against HRH4 to guide further purification. The active fraction was further purified two more times using the same Polar C18 column with extended flushes at 0% B, as the activity always was eluting in the void. A HILIC method proved to be less effective. A single resulting fraction retained activity and this compound was identified as cadaverine by NMR and HRMS (LC-HRMS-ESI (m/z): [M+H]+ calcd for C5H14N2, 102.1157; found 102.12293). Co-eluted in this fraction was the compound agmatine (LC-HRMS-ESI (m/z): [M+H]+ calcd for C5H14N4, 130.12184; found 131.12920). Cadaverine: 1H NMR (D20, 600 MHz): δH 3.04 (4H, t, J=7.6 Hz), 1.74 (4H, p, J=7.7 Hz), 1.49 (2H, p, J=7.7 Hz). 13C NMR (D20, 151 MHz): δC 39.2 (2C, s), 26.2 (2C, s), 22.7 (1C, s).
9,10-Methylenehexadecanoic Acid

Fraction 9 of E. coli LF-82 was injected in DMSO onto a semi-preparative 150×10 mm XBridge® 5 μm C18 column with a solvent system where Solvent A was H₂O+0.1% formic acid and Solvent B was CH3CN+0.1% formic acid. The chromatographic method was as follows: 30% B for 3 column CV then up to 99% B over 5 CV, with a 15 CV hold at 99% B. Peak detection and fraction collection was driven by charged aerosol detection using a Corona Veo (ThermoFisher Scientific) after UV proved to not be useful. Fractions were collected and re-assayed against BAI1 to guide further purification. A single resulting fraction retained activity and this compound was identified as 9,10-methylenehexadecanoic acid by NMR and HRMS (LC-HRMS-ESI (m/z): [M+H]+ calcd for C17H3202, 267.2402; found 267.2334). 9,10-methylenehexadecanoic acid: 1H NMR (CDC13, 600 MHz): δH 2.35 (2H, t, J=7.4 Hz), 1.64 (2H, p, J=7.4 Hz), 1.37 (16H, m), 1.32 (2H, m), 1.14 (2H, m), 0.89 (3H, t, J=6.6 Hz), 0.65 (2H, m), 0.57 (1H, td, J=8.2 Hz, 4.2 Hz), -0.33 (1H, q, J=5.2, 4.4 Hz). 13C NMR (CDC13, 151 MHz): δC 177.7 (1C, s), 33.8 (1C, s), 32.2 (1C, s), 30.4 (1C, s), 30.4 (1C, s), 29.7 (1C, s), 29.6 (1C, s), 29.5 (1C, s), 29.3 (1C, s), 29.0 (1C, s), 28.9 (1C, s), 25.0 (1C, s), 23.0 (1C, s), 16.0 (1C, s), 16.0 (1C, s), 14.4 (1C, s), 11.2 (1C, s).
12-Methylmyristic Acid

Fraction 9 of B. vulgatus was injected in DMSO onto a semi-preparative 150×10 mm XBridge® 5 μm C18 column with a solvent system where Solvent A was H2O+0.1% formic acid and Solvent B was CH3CN+0.1% formic acid. The chromatographic method was as follows: 30% B for 3 column CV then up to 99% B over 5 CV, with a 15 CV hold at 99% B. Peak detection and fraction collection was driven by charged aerosol detection using a Corona Veo (ThermoFisher Scientific) after UV proved to not be useful. Fractions were collected and re-assayed against BAI1 to guide further purification. A single resulting fraction retained activity and this compound was identified as 9,10-methylenehexadecanoic acid by NMR and HRMS. (LC-HRMS-ESI (m/z): [M−H]−calcd for C15H3002, 241.2245; found 241.2178). 12-methylmyristic acid: 1H NMR (CDC13, 600 MHz): δH 2.35 (2H, t, 7.5 Hz), 1.64 (2H, p, 7.5 Hz), 1.26 (16H, m), 1.12 (1H, m, 6.9 Hz), 1.08 (2H, m), 0.85 (3H, t, 7.4 Hz), 0.84 (3H, d, 5.1 Hz). 13C NMR (CDC13, 151 MHz): δC 178.2 (1C, s), 36.6 (1C, s), 34.3 (1C, s), 33.7 (1C, s), 30.0 (1C, s), 29.6 (1C, s), 29.5 (1C, s), 29.4 (1C, s), 29.4 (1C, s), 29.2 (1C, s), 29.0 (1C, s), 27.0 (1C, s), 24.6 (1C, s), 19.2 (1C, s), 11.4 (1C, s).

### Example 2

### Effects of Cadaverine

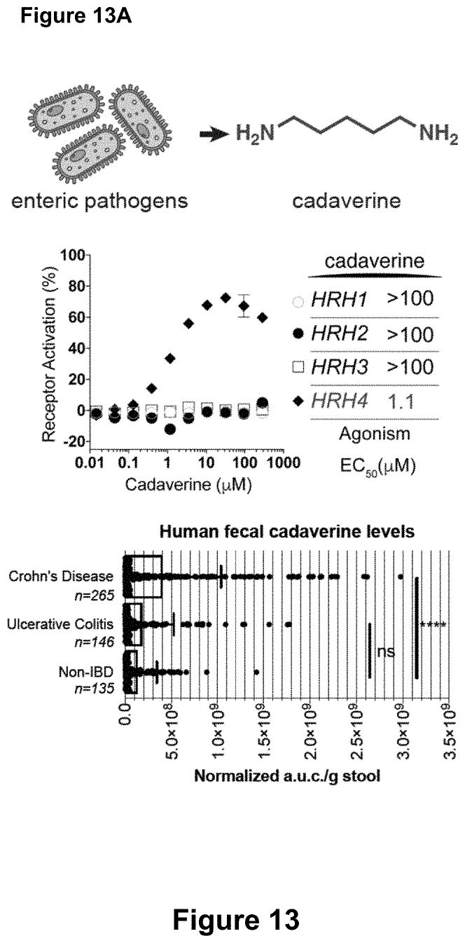

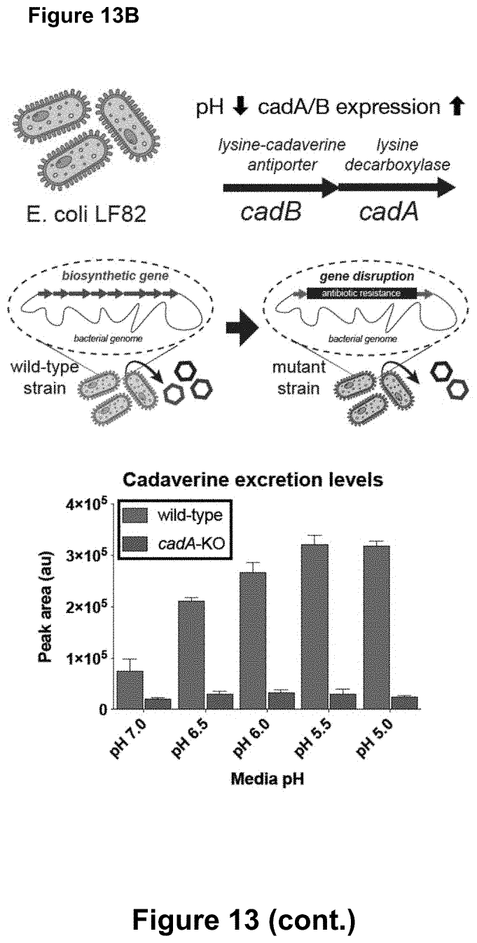

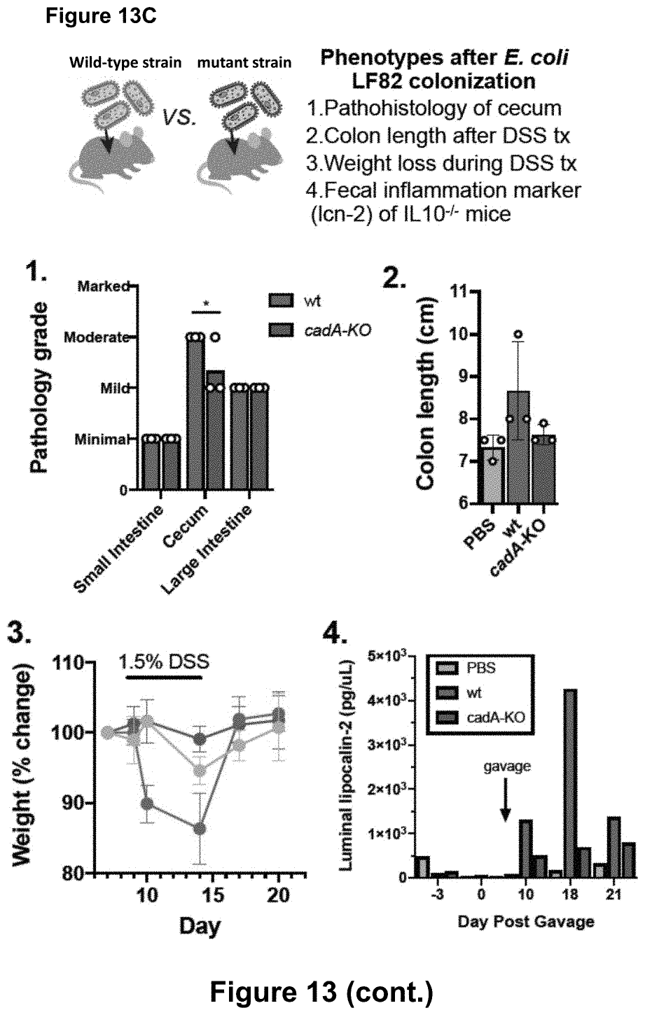

Cadaverine was identified to be an HRH4 agonist that is secreted by pathogenic bacteria and influences gut inflammation (FIG. 13). More specifically, cadaverine was demonstrated to agonize inflammatory receptor and was increased in CD patients (FIG. 13A). Furthermore, cadaverine production was observed to be regulated by acid response and could be genetically decreased (FIG. 13B). CadA was also shown to influence colitis phenotypes in vivo. As shown in FIG. 13C, cadA knockout (KO) mice displayed improved pathohistology of cecum and weight as well as decreased colon length and fecal inflammation.

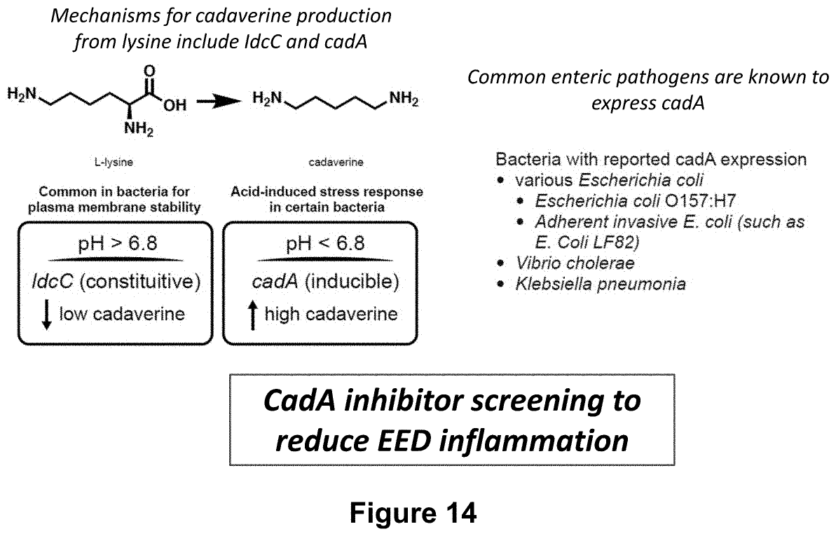

The present study also demonstrated that cadaverine production was dictated by taxa specific cadA gene, which was prevalent in pathogenic bacteria. Moreover, cadaverine is generated from L-lysine in the presence of IdcC and cadA, and common enteric pathogens, such as various Escherichia coli, Escherichia coli 0157:H7, Adherent invasive E. coli (such as E. Coli F82), Vibrio cholerae, and Klebsiella pneumonia, expressed cadA (FIG. 14).

The importance of fatty acids as bacterial bioactives was also examined in the present study (FIG. 15). As shown in FIG. 15A, GPCR screen indicated fatty acids were broadly GPCR active and that certain GPCRs were highly responsive to fatty acid-rich fractions of bacteria and. Moreover, specific lipids selectively activated specific GPCRs. Thus, bacterial specific lipids can help explain health discrepancies in human microbiome and fatty acid content varied between gut bacteria.

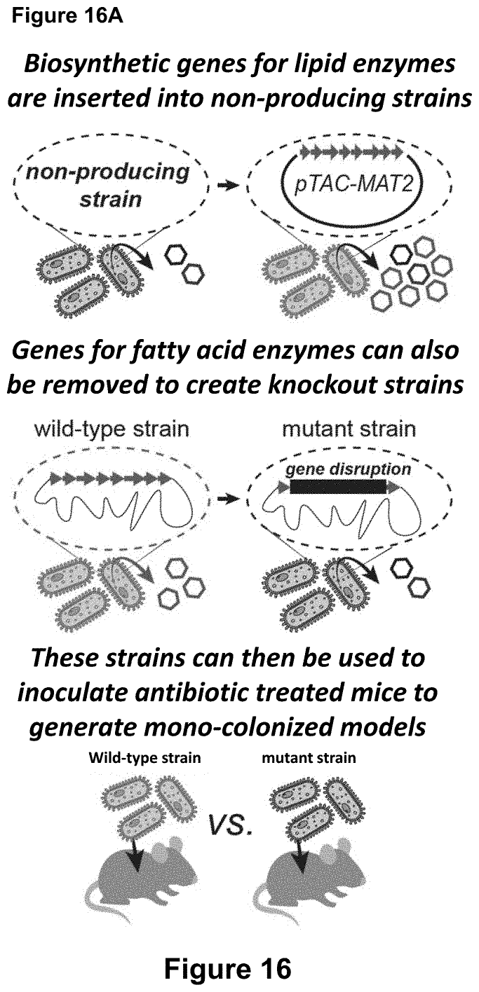

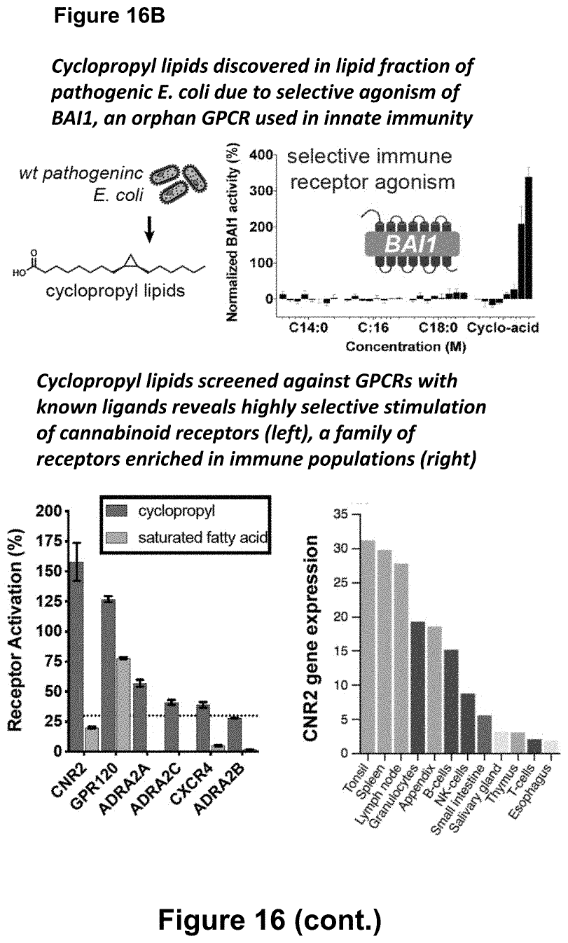

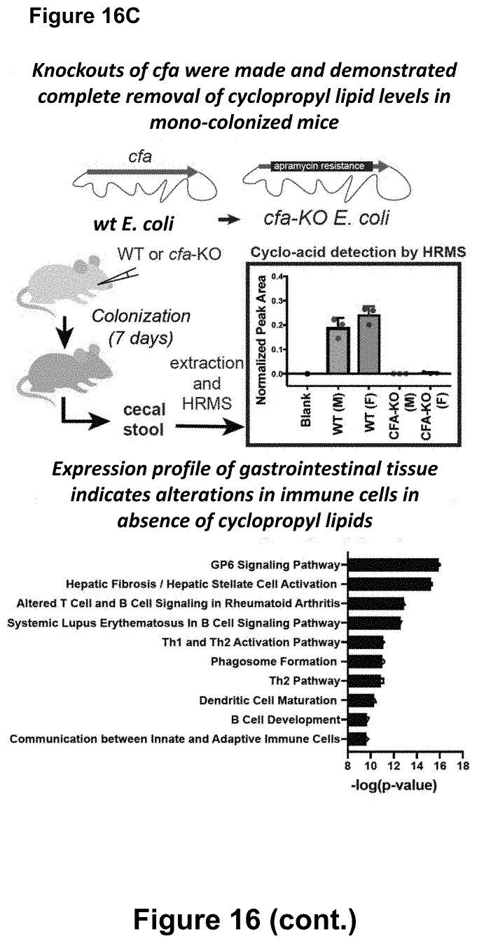

Furthermore, murine models demonstrated effects of taxa-specific fatty acids. Biosynthesis genes for lipid enzymes were inserted into non-producing strains and genes for fatty acid enzymes were also be removed to generate knockout strains. These strains were then used to inoculate antibiotic treated mice to generate mono-colonized models (FIG. 16A). Cyclopropyl lipids agonized orphan and established immune GPCRs. More specifically, cyclopropyl lipids were discovered in lipid fraction of pathogenic E. coli due to selective agonism of BAIL an orphan GPCR used in innate immunity, and cyclopropyl lipids screened against GPCRs with known ligands revealed highly selective stimulation of cannabinoid receptors (FIG. 16B bottom left), a family of receptors enriched in immune populations (FIG. 16B bottom right). In addition, knockouts of cfa were made and demonstrated complete removal of cyclopropyl lipid levels in mono-colonized mice. Expression profile of gastrointestinal tissue also indicated alterations in immune cells in absence of cyclopropyl lipids (FIG. 16C).

### Example 3

### Selective Targeting of GPCR using Microbial Metabolites

TABLE 1
List of Microbial Metabolites and Their Corresponding GPCR

| Ligand | GPCR | UniprotID | EC₅₀ (μM) |
|---|---|---|---|
| 12-methylmyristic acid | GPR151 | Q8TDV0 | 49 |
| 12-methylmyristic acid | UTR2 | Q9UKP6 | 49 |
| 12-methylmyristic acid | NMU1R | Q9HB89 | 29 |
| 13-methylmyristic acid | GPR151 | Q8TDV0 | 44 |
| 14-methylpalmitic acid | GPR151 | Q8TDV0 | 40 |
| 4-hydroxycinnamic acid | GPR109B | P49019 | 208 |
| 9,10-methylenehexadecanoic acid | BAI1 | O14514 | 3.2 |
| 9,10-methylenehexadecanoic acid | CNR2 | P34972 | — |
| 9,10-methylenehexadecanoic acid | UTR2 | Q9UKP6 | 52 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A therapeutic composition comprising a microorganism and/or a microbial metabolite thereof, wherein the microorganism is selected from the group consisting of: Escherichia coli LF82, Enterococcus faecalis, Lactobacilhs plantarum, Faecalibacterium prauznitzii, Bifidobacterium longum, Bacteroides vulgatus, Ruminococcus gnavus, and any combination thereof.

2. The therapeutic composition of claim 1, wherein the microorganism and/or the microbial metabolite thereof binds to a receptor, wherein the receptor is at least one receptor selected from the group consisting of at least one receptor listed in FIG. 2B, at least one receptor listed in FIG. 15, G-protein coupled receptor (GPCR), nuclear receptor (NR), and any combination thereof.

3. (canceled)

4. The therapeutic composition of claim 1, wherein the microorganism and/or the microbial metabolite thereof modulates an activity level of a receptor by at least about 30%.

5. The therapeutic composition of claim 4,
wherein the microorganism and/or the microbial metabolite thereof increases the activity level of the receptor by at least about 30%; or
wherein the microorganism and/or the microbial metabolite thereof inhibits the activity level of the receptor by at least about 30%.

6. (canceled)

7. The therapeutic composition of claim 1, wherein the microbial metabolite is at least one selected from the group consisting of a polypeptide, a carbohydrate, an oligosaccharide, a polysaccharide, a polynucleotide, a lipid, a phospholipid, a fatty acid, a steroid, a peptide, and an amino acid.

8. The therapeutic composition of claim 1, wherein the microbial metabolite is at least one selected from the group consisting of tyramine, tryptamine, cadaverine, 9,10-methylenehexadecanoic acid, 12-methyltetradecanoic acid, 13-methyltetradecanoic acid, 14-methylpalmitic acid, 4-hydroxycinnamic acid, anteiso-fatty acid, and iso-fatty acid.

9. The therapeutic composition of claim 8, wherein
a) tyramine binds to DRD1, DRD2L, DRD2S, DRD3, DRD4, and DRD5;
b) tryptamine binds to HTR1A, HTR1B, HTR1F, HTR1E, HTR2A, HTR2C, and HTR5A;
c) cadaverine binds to HRH1, HRH2, HRH3, and HRH4;
d) 9,10-methylenehexadecanoic acid binds to BAIL CNR2, UTR2;
e) 12-methyltetradecanic acid binds to NMU1R, GPR151, UTR2, ADCYAP1R1;
f) 13-methyltetradecanic acid binds to GPR151;
g) 14-methylpalmitic acid binds to GPR151;
h) 4-hydroxycinnamic acid binds to GPR109B;
i) anteiso-fatty acids bind to NMU1R, UTR2, and GPR120; and
j) iso-fatty acids bind to NMU1R, UTR2, and GPR120.

10. The therapeutic composition of claim 1, wherein the therapeutic composition is used to prevent or treat a disease or disorder in a subject in need thereof

11. The therapeutic composition of claim 1, wherein the disease or disorder is associated with dysfunction of at least one receptor listed in FIG. 2B, FIG. 15, or any combination thereof.

12. A method of treating or preventing a disease or disorder in a subject in need thereof, the method comprising administering the therapeutic composition of claim 1 to the subject.

13. The method of claim 12, wherein the disease or disorder is associated with dysfunction of a receptor listed in FIG. 2B, FIG. 15, or any combination thereof

14. A method of treating or preventing a disease or disorder in a subject in need thereof, the method comprising administering a genetically engineered cell to the subject,
wherein the genetically engineered cell expresses a metabolite that binds to a receptor, wherein the receptor is at least one receptor listed in FIG. 2B, FIG. 15, or any combination thereof.

15. The method of claim 14, wherein the genetically engineered cell is selected from the group consisting of: Escherichia coli LF82, Enterococcus faecalis, Lactobacilhs plantarum, Faecalibacterium prauznitzii, Bifidobacterium longum, Bacteroides vulgatus, Ruminococcus gnavus, and any combination thereof.

16. The method of claim 14, wherein the genetically engineered cell is capable of producing a microbial metabolite selected from the group consisting of tyramine, tryptamine, cadaverine, 9,10-methylenehexadecanoic acid, 12-methyltetradecanoic acid, 13-methyltetradecanoic acid, 14-methylpalmitic acid, 4-hydroxycinnamic acid, anteiso-fatty acid, iso-fatty acid, and any combination thereof.

17. A method of treating or preventing a disease or disorder associated with at least one microorganism and/or a microbial metabolite in a subject in need thereof, the method comprising modulating the level of the at least one microorganism and/or a microbial metabolite thereof in the subject,
wherein the microorganism is selected from the group consisting of: Escherichia coli LF82, Enterococcus faecalis, Lactobacilhs plantarum, Faecalibacterium prauznitzii, Bifidobacterium longum, Bacteroides vulgatus, Ruminococcus gnavus, and any combination thereof.

18. The method of claim 17, wherein the method comprises reducing the level of the at least one microorganism and/or a microbial metabolite thereof in the subj ect.

19. The method of claim 18, wherein the method comprises administering a therapeutically effective amount of an inhibitor of the at least one microorganism and/or microbial metabolite thereof to the subject.

20. The method of claim 19, wherein the inhibitor of the at least one microbial metabolite is an inhibitor of cadaverine, an inhibitor of cadaverine biosynthesis, or a combination thereof.

21. The method of claim 20, wherein the disease or disorder associated with at least one microorganism and/or microbial metabolite is a Crohn's disease.

22. The method of claim 17, wherein the method comprises increasing the level of the at least one microorganism and/or microbial metabolite thereof in the subject.


SEQUENCE LISTING (14 sequences)

SEQ ID NO: 1 - DNA, 21 bp, Artificial Sequence
  Escherichia coli LF-82 (F)
gttaatacctttgctcattga

SEQ ID NO: 2 - DNA, 21 bp, Artificial Sequence
  Escherichia coli LF-82 (R)
accagggtatataatcctgtt

SEQ ID NO: 3 - DNA, 17 bp, Artificial Sequence
  Faecalibacterium prausnitzii DSM 17677 (F)
cccttcagtgccgcagt

SEQ ID NO: 4 - DNA, 18 bp, Artificial Sequence
  Faecalibacterium prausnitzii DSM 17677 (R)
gtcgcaggatgtcaagac

SEQ ID NO: 5 - DNA, 24 bp, Artificial Sequence
  Enterococcus faecalis OG1RF (F)
cccttattgttagttgccatcatt

SEQ ID NO: 6 - DNA, 21 bp, Artificial Sequence
  Enterococcus faecalis OG1RF (R)
actcgttgtacttcccattgt

SEQ ID NO: 7 - DNA, 18 bp, Artificial Sequence
  Bifidobacterium longum ATCC 15707 (F)
gggtggtaatgccggatg

SEQ ID NO: 8 - DNA, 21 bp, Artificial Sequence
  Bifidobacterium longum ATCC 15707 (R)
taagcgatggactttcacacc

SEQ ID NO: 9 - DNA, 19 bp, Artificial Sequence
  Lactobacillus plantarum ATCC BAA-793 (F)
agcagtagggaatcttcca

SEQ ID NO: 10 - DNA, 17 bp, Artificial Sequence
  Lactobacillus plantarum ATCC BAA-793 (R)
caccgctacacatggag

SEQ ID NO: 11 - DNA, 20 bp, Artificial Sequence
  Bacteroides vulgatus ATCC 8482 (F)
ggtgtcggcttaagtgccat

SEQ ID NO: 12 - DNA, 18 bp, Artificial Sequence
  Bacteroides vulgatus ATCC 8482 (R)
cggaygtaagggccgtgc

SEQ ID NO: 13 - DNA, 19 bp, Artificial Sequence
  Ruminococcus gnavus ATCC 29149 (F)
cggtacctgactaagaagc

SEQ ID NO: 14 - DNA, 19 bp, Artificial Sequence
  Ruminococcus gnavus ATCC 29149 (R)
agtttyattcttgcgaacg